(19) United States
(12) Patent Application Publication
(10) Pub. No.: US 2024/0000939 A1
(43) Pub. Date: Jan. 4, 2024

(54) METHODS OF PERSONALIZED PRECONDITIONING FOR CELL THERAPY

(71) Applicant: MEMORIAL SLOAN-KETTERING CANCER CENTER, New York, NY (US)

(72) Inventors: Jaap Jan Boelens, New York, NY (US); Kevin J. Curran, Fairfield, CT (US)

(73) Assignee: MEMORIAL SLOAN-KETTERING CANCER CENTER, New York, NY (US)

(21) Appl. No.: 18/368,647

(22) Filed: Sep. 15, 2023

Related U.S. Application Data
(63) Continuation of application No. PCT/US2022/020489, filed on Mar. 16, 2022.
(60) Provisional application No. 63/161,724, filed on Mar. 16, 2021.

(51) Int. Cl. A61K 39/00; C12N 5/0783; A61K 31/7076; A61P 35/02

(52) U.S. Cl. CPC A61K 39/464412; C12N 5/0636; A61K 39/4622; A61K 39/4631; A61K 39/464411; A61K 39/4611; A61K 31/7076; A61P 35/02; A61K 2239/11; A61K 2239/22; A61K 2239/48

(57) ABSTRACT

The present disclosure relates to treating a subject comprising administering to the subject a therapy (e.g., a cell therapy, e.g., an adoptive cell therapy, e.g., a CAR-T cell therapy), wherein, prior to the administration, the subject has been preconditioned with a personalized amount of a chemotherapeutic agent. The personalized amount provides an optimal exposure to the chemotherapeutic agent.

### CROSS-REFERENCE TO RELATED APPLICATIONS

This application is a Continuation application of International Patent Application No. PCT/US2022/020489, filed Mar. 16, 2022, which claims priority to U.S. Provisional Application No. 63/161,724, filed Mar. 16, 2021, the contents of each of which are incorporated by reference in their entirety herein and to each of which priority is claimed.

### 1. TECHNICAL FIELD

The present disclosure relates to methods for treating a subject comprising administering to the subject a therapy (e.g., a cell therapy, e.g., an adoptive cell therapy, e.g., a CAR-T cell therapy), wherein, prior to the administration, the subject has been preconditioned with a personalized amount of a chemotherapeutic agent. The personalized amount provides an optimal exposure to the chemotherapeutic agent.

### 2. BACKGROUND

Chimeric antigen receptor (CAR) T-cells have provided a therapeutic option for patients with relapsed or refractory (R/R) B-cell acute lymphoblastic leukemia (B-ALL) who have failed standard chemotherapy approaches. Under the current medical practice, a lymphodepleting round of chemotherapy prior to CAR T-cell injections (i.e., preconditioning) can increase the efficacy of the CAR T-cell treatment. By reducing tumor burden and the number of normal immune cells, chemotherapy allows CAR-T cells to proliferate and overcome the cancer cells more easily.

However, the current dosing regimen of chemotherapy, which is given based on the body surface area of patients, leads to highly variable fludarabine exposures and failure to consistently improve the efficacy of CAR-T cell therapy and achieve optimal outcomes in all patients. Despite initial response, the incidence of relapse and death still approaches 50% in CAR-T cell treated patients.

Accordingly, there is unmet need for improved preconditioning methods that provides optimal efficacy of CAR-T cell therapy in patients.

### 3. SUMMARY OF THE INVENTION

The present disclosure relates to methods for treating a subject comprising administering to the subject a therapy (e.g., a cell therapy, e.g., an adoptive cell therapy, e.g., a CAR-T cell therapy), wherein, prior to the administration, the subject has been preconditioned with a personalized amount of a chemotherapeutic agent. The personalized amount provides an optimal exposure to the chemotherapeutic agent. In certain embodiments, the optimal exposure is indicated by an area under the curve (AUC).

In certain embodiments, the present disclosure provides a method of treating a subject, comprising administering to the subject cells comprising an antigen recognizing receptor, wherein, prior to the administration, the subject has been preconditioned with a personalized amount of a chemotherapeutic agent, wherein the personalized amount provides an area under the curve (AUC) of at least about 10 mg·hr/L of the chemotherapeutic agent. In certain embodiments, the personalized amount is not greater than about 40 mg·hr/L of the chemotherapeutic agent.

In certain embodiments, the present disclosure provides a method of increasing durability of a therapy comprising cells comprising an antigen recognizing receptor in a subject, the method comprising: administering to the subject the therapy, wherein, prior to the administration, the subject has been preconditioned with a personalized amount of a chemotherapeutic agent, wherein the personalized amount provides an area under the curve (AUC) of at least about 10 mg·hr/L of the chemotherapeutic agent. In certain embodiments, the personalized amount is not greater than about 40 mg·hr/L of the chemotherapeutic agent.

In certain embodiments, the present disclosure provides a method of improving efficacy of a therapy comprising cells comprising an antigen recognizing receptor in a subject, the method comprising administering to the subject the therapy, wherein, prior to the administration, the subject has been preconditioned with a personalized amount of a chemotherapeutic agent, wherein the personalized amount provides an area under the curve (AUC) of at least about 10 mg·hr/L of the chemotherapeutic agent. In certain embodiments, the personalized amount is not greater than about 40 mg·hr/L of the chemotherapeutic agent.

In certain embodiments, the present disclosure provides a method of lowering the risk of relapse and/or lengthening survival of a subject receiving a therapy comprising cells comprising an antigen recognizing receptor, the method comprising preconditioning the subject with a personalized amount of a chemotherapeutic agent prior to the therapy, wherein the personalized amount provides an area under the curve (AUC) of at least about 10 mg·hr/L of the chemotherapeutic agent. In certain embodiments, the personalized amount is not greater than about 40 mg·hr/L of the chemotherapeutic agent.

In certain embodiments, the personalized amount of the chemotherapeutic agent provides an AUC of at least about 11 mg·hr/L, at least about 12 mg·hr/L, at least about 13 mg·hr/L, at least about 14 mg·hr/L, at least about 15 mg·hr/L, or at least about 20 mg·hr/L of the chemotherapeutic agent. In certain embodiments, the personalized amount of the chemotherapeutic agent provides an AUC of at least about 13 mg·hr/L of the chemotherapeutic agent. In certain embodiments, the personalized amount of the chemotherapeutic agent provides an AUC of about 14 mg·hr/L of the chemotherapeutic agent.

In certain embodiments, the personalized amount is determined by body weight and renal function of the subject. In certain embodiments, the renal function is determined by a glomerular filtration rate (GFR) of the subject. In certain embodiments, the GFR is determined by creatinine clearance of the subject.

In certain embodiments, the chemotherapeutic agent is an antimetabolite. In certain embodiments, the antimetabolite is selected from the group consisting of folic acid antagonists, pyrimidine analogs, purine analogs, adenosine deaminase inhibitors, lympho- or myeloid depleting antibodies (e.g., monoclonal antibodies, polyclonal antibodies, bispecific antibodies), alkylators, topoisornerae 11 inhibitors, derivatives thereof, and combinations thereof In certain embodiments, the chemotherapeutic agent is an adenosine deaminase inhibitor. In certain embodiments, the adenosine deaminase inhibitor is fludarabine or a derivative thereof. In certain embodiments, the chemotherapeutic agent is a purine analog. In certain embodiments, the purine analog is clofarabine or a derivative thereof.

In certain embodiments, the subject suffers a tumor. In certain embodiments, the tumor is cancer. In certain embodiments, the tumor is blood cancer. In certain embodiments, the tumor is B-cell malignancy. In certain embodiments, the tumor is leukemia or lymphoma. In certain embodiments, the tumor is selected from the group consisting of B cell leukemia, B cell lymphoma, acute lymphoblastic leukemia (ALL), chronic lymphocytic leukemia (CLL), non-Hodgkin's lymphoma, Burkitt lymphoma, acute myeloid leukemia (AML) and Mixed-phenotype acute leukemia (VIPAL). In certain embodiments, the tumor is B cell acute-lymphoblastic leukemia (B-ALL).

In certain embodiments, the subject has a high disease burden at the time of or immediately prior to the preconditioning. In certain embodiments, the subject who has high disease burden has more than about 5% lymphoblasts in bone marrow, detectable peripheral blood lymphoblasts in the subject, a CNS3 status, and/or non-CNS extramedullary (EM) site of disease. In certain embodiments, the CNS3 status is determined by detecting at least about 5 white blood cells/ml cerebrospinal fluid and at least one lymphoblast in the cerebrospinal fluid.

In certain embodiments, the antigen-recognizing receptor is a T cell receptor (TCR) or a chimeric antigen receptor (CAR). In certain embodiments, the antigen-recognizing receptor is a CAR. In certain embodiments, the CAR comprises an extracellular antigen-recognition domain and an intracellular signaling domain. In certain embodiments, the extracellular antigen-binding domain binds to an antigen. In certain embodiments, the antigen is a tumor antigen or a pathogen antigen. In certain embodiments, the antigen is CD19. In certain embodiments, the intracellular signaling domain comprises a CD3 polypeptide. In certain embodiments, the intracellular signaling domain of the CAR further comprises at least one co-stimulatory signaling region. In certain embodiments, the costimulatory signaling region comprises an intracellular domain of 4-1BB or an intracellular domain of CD28.

In certain embodiments, the cell is an immunoresponsive cell. In certain embodiments, the cell is a cell of the lymphoid lineage or a cell of the myeloid lineage. In certain embodiments, the cell is selected from the group consisting of T cells, Natural Killer (NK) cells, stem cells from which lymphoid cells may be differentiated. In certain embodiments, the cell is a T cell. In certain embodiments, the T cell is selected from the group consisting of a cytotoxic T lymphocyte (CTL), a γ6 T cell, a tumor-reactive lymphocyte, a tumor-infiltrating lymphocyte (TIL), a regulatory T cell, and a Natural Killer T (NKT) cell.

### 4. BRIEF DESCRIPTION OF THE DRAWINGS

FIG. 1 shows the analysis of the impact of fludarabine AUC on cumulative incidence of relapse (CIR).

FIGS. 2A and 2B show the comparison between patient who had fludarabine AUC of ≥13.8 mg·hr/L, and patients who had fludarabine AUC of <13.8 mg·hr/L. Cumulative Incidence of Relapse (FIG. 2A) and Composite Endpoint (Loss of BCA or Relapse) (FIG. 2B) were measured.

FIGS. 3A-3B show the comparison between patient who had a fludarabine AUC of ≥13.8 mg·hr/L, and patients who had a fludarabine AUC of <13.8 mg·hr/L. All patients had high pre-treatment disease burden. Overall Survival (FIG. 3A) and Cumulative Incidence of Relapse (FIG. 3B) in patient having high pre-treatment disease burden were measured.

FIG. 4 shows the patient flow diagram of the study design described in Example 2.

FIGS. 5A-5D show graphs reporting the impact of fludarabine exposure and pre-infusion disease burden on outcomes after tisagenlecleucel in responding patients. Patients were cohorted based on fludarabine exposure (optimal vs sub-optimal) and pre-infusion disease burden (high vs low/no). FIG. 5A shows cumulative incidence of relapse of the cohorted responders to tisagenlecleucel. FIG. 5B shows cumulative incidence of composite endpoint of the cohorted responders to tisagenlecleucel. FIG. 5C shows OS of all responding patients after tisagenlecleucel. FIG. 5D shows the impact of fludarabine exposure and pre-infusion disease burden on overall survival in all treated patients after tisagenlecleucel infusion.

### 5. DETAILED DESCRIPTION

The present disclosure relates to methods of treating a subject, wherein the method comprises administering to the subject a therapy, and prior to the administration, the subject has been preconditioned with a personalized amount of a chemotherapeutic agent. The preconditioning with the personalized amount of the chemotherapeutic agent can improve the efficacy of the therapy. The therapy can be a cell therapy, e.g., an adoptive cell therapy, e.g., a therapy comprising cells (e.g., T cells) comprising an antigen-recognizing receptor (e.g., a chimeric antigen receptor (CAR) or a T cell receptor (TCR). In certain embodiments, the chemotherapeutic agent comprises fludarabine. The present disclosure is at least based on the discovery that patients who have been pre-conditioned with an optimal fludarabine exposure (as indicated by an area under the curve (AUC)) achieved improved efficacy of a CAR-T cell therapy, such as reduced relapse, maintained B cell aplasia, increased durability of the CAR T-cell therapy, and increased survival. The beneficial effects of the optimal fludarabine exposure was also observed in patients who had high disease burden prior to the CAR T-cell therapy.

Non-limiting embodiments of the present disclosure are described by the present specification and Examples.

For purposes of clarity of disclosure and not by way of limitation, the detailed description is divided into the following subsections:
5.1. Definitions;
5.2. Methods of Treatment;
5.3. Chemotherapeutic Agents;
5.4. Therapy; and
5.5 Administration.

### Definitions

Unless defined otherwise, all technical and scientific terms used herein have the meaning commonly understood by a person skilled in the art. The following references provide one of skill with a general definition of many of the terms used in the presently disclosed subject matter: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

By “immunoresponsive cell” is meant a cell that functions in an immune response or a progenitor, or progeny thereof. In certain embodiments, the immunoresponsive cell is a cell of lymphoid lineage. Non-limiting examples of cells of lymphoid lineage include T cells, Natural Killer (NK) cells, B cells, and stem cells from which lymphoid cells may be differentiated. In certain embodiments, the immunoresponsive cell is a cell of myeloid lineage.

The term “antigen-recognizing receptor” as used herein refers to a receptor that is capable of activating an immunoresponsive cell (e.g., a T cell) in response to its binding to an antigen.

The term “chimeric antigen receptor” or “CAR” as used herein refers to a molecule comprising an extracellular antigen-binding domain that is fused to an intracellular signaling domain that is capable of activating an immunoresponsive cell, and a transmembrane domain. In certain embodiments, the extracellular antigen-binding domain of a CAR comprises a single chain variable fragment (scFv). The scFv can be derived from fusing the variable heavy and light regions of an antibody. Alternatively or additionally, the scFv may be derived from Fab' s (instead of from an antibody, e.g., obtained from Fab libraries). In certain embodiments, the scFv is fused to the transmembrane domain and then to the intracellular signaling domain.

An “effective amount” is an amount sufficient to affect a beneficial or desired clinical result upon treatment. An effective amount can be administered to a subject in one or more doses. In certain embodiments, an effective amount can be an amount that is sufficient to palliate, ameliorate, stabilize, reverse or slow the progression of the disease, or otherwise reduce the pathological consequences of the disease. The effective amount can be determined by a physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

By “increase” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

The term “antigen-binding domain” as used herein refers to a domain capable of specifically binding a particular antigenic determinant or set of antigenic determinants present on a cell.

The terms “comprises”, “comprising”, and are intended to have the broad meaning ascribed to them in U.S. Patent Law and can mean “includes”, “including” and the like.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys. The term “immunocompromised” as used herein refers to a subject who has an immunodeficiency. The subject is very vulnerable to opportunistic infections, infections caused by organisms that usually do not cause disease in a person with a healthy immune system but can affect people with a poorly functioning or suppressed immune system.

### 5.2. Methods of Treatment

The present disclosure provides methods for treating a subject, for improving the efficacy of a therapy, for improving the durability of a therapy, and for maintaining B-cell aplasia in a subject receiving a therapy. In certain embodiments, the therapy is an adoptive cell therapy. In certain embodiments, the methods disclosed herein comprise administering to the subject a therapy (e.g., an adoptive cell therapy), wherein, prior to the administration, the subject has been preconditioned with a personalized amount of a chemotherapeutic agent.

B-cell aplasia, defined as “low numbers of B cells or absent B cells”, can be measured for its ability to predict treatment efficacy and persistence for an adoptive cell therapy (in setting of CD19 targeted therapy), e.g., a CAR-T cell therapy. In CD19 CAR T cell trials, B cell aplasia has been shown to be a treatable and tolerable toxicity. Furthermore, B cell aplasia can be a marker of CAR T cell efficacy and persistence, and thus an indicator of treatment success and risk of relapse (Maude et al., Blood (2015);125 (26):4017-23). In certain embodiments, the B-cell aplasia indicates the existence of durable functional CAR T-cells in the subject. In certain embodiments, the B-cell aplasia indicates less than about 1% B cells of peripheral blood mononuclear cells of a subject. In certain embodiments, the B-cell aplasia indicates less than about 1% CD19⁺CD²⁰⁺B cells of peripheral blood mononuclear cells of a subject.

In certain embodiments, the presence and/or persistence of CAR-T cells is measured by antibodies that binds to the antigen to which the CAR binds.

In certain embodiments, the present disclosure provides methods for treating and/or preventing a tumor in a subject. In certain embodiments, the present disclosure provides methods for reducing disease burden in a subject having a tumor. In certain embodiments, the methods disclosed herein comprise administering to the subject a therapy, wherein, prior to the administration, the subject has been preconditioned with a personalized amount of a chemotherapeutic agent. In certain embodiments, the therapy is an adoptive cell therapy.

In certain embodiments, reducing the disease burden comprises reducing a total number of tumor cells in an organ, tissue, or bodily fluid of the subject, such as the organ or tissue of the tumor or another location, e.g., which would indicate metastasis. In certain embodiments, reducing the disease burden comprises reducing the total number of tumor cells detected and/or quantified in the blood or bone marrow in the context of certain hematological malignancies. In certain embodiments, reducing the disease burden comprises reducing the number of tumor cells, reducing tumor size, reducing tumor mass, and/or reducing the number or extent of metastases. In certain embodiments, the subject has leukemia, and reducing the disease burden comprises reducing residual leukemia in blood or bone marrow (and cerebral spinal fluid) and/or reducing the percentage of lymphoblast cells present in bone marrow.

In certain embodiments, the present disclosure provides methods for lowering the risk of relapse, lengthening survival, and/or promoting event (e.g., cancer)-free survival in a subject receiving a therapy. In certain embodiments, the methods disclosed herein comprise administering to the subject the therapy, wherein, prior to the administration, the subject has been preconditioned with a chemotherapeutic agent in a personalized amount. In certain embodiments, the therapy is an adoptive cell therapy.

Non-limiting examples of tumors include blood cancer, B cell leukemia, B cell lymphoma, acute lymphoblastic leukemia (ALL), chronic lymphocytic leukemia (CLL), acute myeloid leukemia (AML), multiple myeloma, lymphoma (Hodgkin's lymphoma, non-Hodgkin's lymphoma), glioblastoma, myelodysplastic syndrome (MDS), and chronic myelogenous leukemia (CIVIL), bone cancer, intestinal cancer, liver cancer, skin cancer, cancer of the head or neck, melanoma (cutaneous or intraocular malignant melanoma), renal cancer (e.g. clear cell carcinoma), throat cancer, prostate cancer (e.g. hormone refractory prostate adenocarcinoma), blood cancers (e.g. leukemias, lymphomas, and myelomas), uterine cancer, rectal cancer, cancer of the anal region, bladder cancer, brain cancer, stomach cancer, testicular cancer, carcinoma of the fallopian tubes, carcinoma of the endometrium, carcinoma of the cervix, carcinoma of the vagina, carcinoma of the vulva, leukemias (e.g., acute leukemia, acute lymphocytic leukemia, acute myelocytic leukemia, acute myeloblastic leukemia, acute promyelocytic leukemia, acute monocytic leukemia, acute erythroleukemia, chronic leukemia, chronic myelocytic leukemia, polycythemia vera, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, include Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, hepatoma, nile duct carcinoma, choriocarcinoma, seminoma, embryonal carcinoma, Wilm's tumor, cervical cancer, salivary gland cancer, uterine cancer, testicular cancer, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). In certain embodiments, the tumor is a cancer.

In certain embodiments, the tumor is blood cancer. In certain embodiments, the tumor is leukemia or lymphoma. In certain embodiments, the tumor is selected from the group consisting of B cell leukemia, B cell lymphoma, acute lymphoblastic leukemia (ALL) (e.g., T cell acute lymphoblastic leukemia (T-ALL), B cell acute lymphoblastic leukemia (B-ALL)), chronic lymphocytic leukemia (CLL) (e.g., T cell chronic lymphocytic leukemia (T-CLL), B cell chronic lymphocytic leukemia (B-CLL)), multiple myeloma, non-Hodgkin's lymphoma, Burkitt lymphoma, acute myeloid leukemia (AML), and Mixed-phenotype acute leukemia (1VIPAL). In certain embodiments, the tumor is B cell acute lymphoblastic leukemia (B-ALL).

In certain embodiments, the present disclosure provides methods for treating or preventing an autoimmune disease. Non-limiting examples of autoimmune disease include Systemic lupus erythematosus (SLE) Rheumatoid arthritis (RA), Juvenile Rheumatoid Arthritis (JIA). In certain embodiments, the present disclosure provides methods for treating or preventing an allo-immune disease, e.g., Graft Versus Host Disease. In certain embodiments, the present disclosure provides methods for treating or preventing an infectious disease.

In certain embodiments, the subject has a high disease burden at the time of or immediately prior to the preconditioning. In certain embodiments, the subject who has a high disease burden has more than about 5% lymphoblasts in bone marrow. In certain embodiments, the subject who has a high disease burden has detectable peripheral blood lymphoblasts. In certain embodiments, the subject who has a high disease burden has a CNS3 status. In certain embodiments, the CNS3 status refers to the detection of at least about five (5) white blood cells/ml cerebrospinal fluid and at least one lymphoblast in the cerebrospinal fluid. In certain embodiments, the subject who has a high disease burden has non-CNS extramedullary (EM) site of disease.

### Preconditioning With a Chemotherapeutic Agent

In certain embodiments, the subject, prior to the administration of the therapy, has been preconditioned with a personalized amount of a chemotherapeutic agent. The presently disclosed personalized amount of the chemotherapeutic agent provides an optimal exposure of the chemotherapeutic agent to the subject. In certain embodiments, the optimal exposure of the chemotherapeutic agent is indicated by an area under the curve (AUC) of the chemotherapeutic agent in the subject.

In certain embodiments, the personalized amount of the chemotherapeutic agent provides an AUC of at least about 10 mg·hr/L. In certain embodiments, the personalized amount of the chemotherapeutic agent provides an AUC of at least about 11 mg·hr/L, at least about 12 mg·hr/L, at least about 13 mg·hr/L, at least about 14 mg·hr/L, at least about 15 mg·hr/L, at least about 16 mg·hr/L, at least about 17 mg·hr/L, at least about 18 mg·hr/L, at least about 19 mg·hr/L, at least about 20 mg·hr/L, at least about 25 mg·hr/L, at least about 30 mg·hr/L, at least about 35 mg·hr/L, or at least about 40 mg·hr/L,. In certain embodiments, the personalized amount of the chemotherapeutic agent provides an AUC of no greater than about 40 mg·hr/L, no greater than about 35 mg·hr/L, no greater than about 30 mg·hr/L, no greater than about 25 mg·hr/L, or no greater than about 20 mg·hr/L. In certain embodiments, the personalized amount of the chemotherapeutic agent provides an AUC of between about 10 mg·hr/L and about 40 mg·hr/L.

In certain embodiments, the personalized amount of the chemotherapeutic agent provides an AUC of at least about 12.0 mg·hr/L, at least about 12.5 mg·hr/L, at least about 13.0 mg·hr/L, at least about 13.5 mg·hr/L, at least about 14.0 mg·hr/L, at least about 14.5 mg·hr/L, or at least about 15.0 mg·hr/L. In certain embodiments, the personalized amount of the chemotherapeutic agent provides an AUC of between about 12.0 mg·hr/L and about 15.0 mg·hr/L, e.g., between about 12.0 mg·hr/L and about 14.0 mg·hr/L, between about 13.0 mg·hr/L and about 15.0 mg·hr/L, between about 13.0 mg·hr/L and about 14.0 mg·hr/L, or between about 13.5 mg·hr/L and about 14.0 mg·hr/L. In certain embodiments, the personalized amount of the chemotherapeutic agent provides an AUC of at least about 14 mg·hr/L. In certain embodiments, the personalized amount of the chemotherapeutic agent provides an AUC of about 14 mg·hr/L. In certain embodiments, the personalized amount of the chemotherapeutic agent provides an AUC of at least about 13.5 mg·hr/L. In certain embodiments, the personalized amount of the chemotherapeutic agent provides an AUC of about 13.5 mg·hr/L. In certain embodiments, the personalized amount of the chemotherapeutic agent provides an AUC of at least 13.8 mg·hr/L. In certain embodiments, the personalized amount of the chemotherapeutic agent provides an AUC of 13.8 mg·hr/L.

In certain embodiments, the chemotherapeutic agent is fludarabine. In certain embodiments, the personalized amount of fludarabine provides an AUC of at least about 14 mg·hr/L. In certain embodiments, the personalized amount of fludarabine provides an AUC of about 14 mg·hr/L. In certain embodiments, the personalized amount of fludarabine provides an AUC of at least about 13.5 mg·hr/L. In certain embodiments, the personalized amount of fludarabine provides an AUC of about 13.5 mg·hr/L. In certain embodiments, the personalized amount of fludarabine provides an AUC of at least 13.8 mg·hr/L. In certain embodiments, the personalized amount of fludarabine provides an AUC of 13.8 mg·hr/L.

In certain embodiments, the personalized amount of the chemotherapeutic agent is determined by body weight and renal function of the subject. In certain embodiments, the renal function is indicated by a glomerular filtration rate (GFR). In certain embodiments, the GFR is an estimated glomerular filtration rate (GFR). In certain embodiments, the GFR or eGFR is indicated by creatinine clearance of the subject. In certain embodiments, the creatinine clearance of the subject is calculated using Cockroft—Gault equation as disclosed in Cockcroft et al., Nephron. 1976;16 (1):31-41, the contents of which are incorporated herein by reference in its entirety. In certain embodiments, the creatinine clearance of the subject is calculated using the following formula:

Creatine Clearance (male)=([140-age]×weight in kg)/(serum creatinine×72)

Creatine Clearance (female)=Creatine Clearance (male)×0.85

In certain embodiments, the GFR is estimated using Cystatin-C. In certain embodiments, the GFR is estimated by performing a nuclear scan.

In certain embodiments, the personalized amount of the chemotherapeutic agent is determined by a population pharmacokinetics model of the chemotherapeutic agent. In certain embodiments, the population pharmacokinetics model is a three-compartment model. In certain embodiments, the population pharmacokinetics model is a one-compartment model. In certain embodiments, the population pharmacokinetics model is a two-compartment model.

In certain embodiments, the population pharmacokinetics model is a three-compartment model, and the chemotherapeutic agent is fludarabine.

In certain embodiments, the three-compartment model is parameterized in terms of volume of distribution of the central (V1), peripheral (V2), and second peripheral (V3) compartment, and clearance from the central compartment as well as intercompartmental clearance between V1 and V2 (Q2) and V1 and V3 (Q3).

In certain embodiments, the chemotherapeutic agent is fludarabine. In certain embodiments, the personalized amount of the chemotherapeutic agent is determined by a population pharmacokinetics model disclosed in Langenhorst et al., Clinical Pharmacokinetics (2019) 58:627-637.

$$Cl = ( {Cl}_{70 kg - non - renal} + eGFR \times {Slope}_{pep} ) \times {( \frac{BW}{70 kg} )}^{0.75} \qquad Structural model$$

Preconditioning the subject with the personalized amount of the chemotherapeutic agent, prior to the administration of the therapy, can improve the efficacy of the therapy, improve the responsiveness of the subject to the therapy, and improve the durability of the therapy. In certain embodiments, the subject has B cell malignancy, and the preconditioning maintains B-cell aplasia of the subject.

### Chemotherapeutic Agents

Any suitable chemotherapeutic agents known in the art can be used in the precondition regimen of the presently disclosed methods. Non-limiting suitable examples of chemotherapeutic agents include antimetabolite, alkylating agents, anthracyclines (e.g., doxorubicin and liposomal doxorubicin), vinca alkaloids (e.g., vinblastine, vincristine, vindesine, and vinorelbine), immune cell antibodies (e.g., alemtuzamab, gemtuzumab, rituximab, ofatumumab, tocitumomab, brentuximab: see below also ATG), mTOR inhibitors (e.g., rapamycin, rapamycin analogs, sirolimus, temsirolimus, everolimus, ridaforolimus, umirolimus, zotarolimus), TNFR glucocorticoid induced TNFR related protein (GITR) agonists (e.g., TRX518), proteasome inhibitors (e.g., aclacinomycin A, gliotoxin or bortezomib), immunomodulators such as thalidomide or thalidomide derivatives (e.g., lenalidomide), cytokine antagonists (e.g. IL1 blockade such as Anakinra), derivatives thereof, and combinations thereof.

In certain embodiments, the chemotherapeutic agent is an antimetabolite. In certain embodiments, the antimetabolite is selected from the group consisting of folic acid antagonists, pyrimidine analogs, purine analogs and adenosine deaminase inhibitors, lympho- or myeloid depleting antibodies (e.g., monoclonal antibodies, polyclonal antibodies, or bispecific antibodies), alkylators, topoisomerase II inhibitors, derivatives thereof, and combinations thereof.

In certain embodiments, the chemotherapeutic agent is a purine analog. In certain embodiments, the purine analog is selected from the group consisting of clofarabine, 6-mercaptopurine and 6-thioguanine, derivatives thereof, and combinations thereof In certain embodiments, the chemotherapeutic agent is clofarabine or a derivative thereof.

In certain embodiments, the chemotherapeutic agent is an adenosine deaminase inhibitor. In certain embodiments, the adenosine deaminase inhibitor is selected from the group consisting of fludarabine, cladribine, pentostatin, derivatives thereof, and combinations thereof In certain embodiments, the chemotherapeutic agent is fludarabine or a derivative thereof.

In certain embodiments, the chemotherapeutic agent is a folic acid antagonist. In certain embodiments, the folic acid antagonist is selected from the group consisting of methotrexate, derivatives thereof, and combinations thereof.

In certain embodiments, the chemotherapeutic agent is a pyrimidine analog. In certain embodiments, the pyrimidine analog is selected from the group consisting of 5-fluorouracil, floxuridine, cytarabine, capecitabine, and gemcitabine, derivatives thereof, and combinations thereof.

In certain embodiments, the chemotherapeutic agent is an alkylating agent. In certain embodiments, the alkylating agent is selected from the group consisting of cyclophosphamide, decarbazine, melphalan, ifosfamide, temozolomide, treosulfan, thiothepa, busulfan, derivatives thereof, and combinations thereof. In certain embodiments, the chemotherapeutic agent is cyclophosphamide or a derivative thereof.

In certain embodiments, the chemotherapeutic agent is an antibody. Non-limiting examples of antibodies include Alemtuzumab (Anti-CD52), rabbit ATG (Thymoglobuline), rabbit ATLG (anti-thymocyte lymphoglobuline), anti-CD30 (Brentuximab).

### 5.4 Therapy

In certain embodiments, the therapy is an immunotherapy. In certain embodiments, the therapy is a cell therapy. In certain embodiments, the therapy is an adoptive cell therapy. In certain embodiments, the therapy cells comprising an antigen-recognizing receptor. In certain embodiments, the antigen-recognizing receptors binds to an antigen. In certain embodiments, the antigen-recognizing receptor is a chimeric antigen receptor (CAR). In certain embodiments, the antigen-recognizing receptor is a T-cell receptor (TCR). The antigen-recognizing receptor can bind to a tumor antigen or a pathogen antigen. In certain embodiments, the antigen-recognizing receptor binds to a tumor antigen. In certain embodiments, the tumor antigen is a tumor-specific antigen or a tumor-associated antigen.

### 5.4.1 Antigens

In certain embodiments, the antigen-recognizing receptor binds to a tumor antigen. Any tumor antigen (antigenic peptide) can be used in the tumor-related embodiments described herein. Sources of antigen include, but are not limited to, cancer proteins. The antigen can be expressed as a peptide or as an intact protein or portion thereof. The intact protein or a portion thereof can be native or mutagenized. In certain embodiments, the tumor antigen is a tumor specific antigen (TSA). In certain embodiment, the tumor antigen is a tumor-associated antigen (TAA).

Non-limiting examples of tumor antigens include CD19, MUC16, MUC1, CAIX, CEA, CD8, CD7, CD10, CD20, CD22, CD30, CLL1, CD33, CD34, CD38, CD41, CD44, CD49f, CD56, CD74, CD123, CD133, CD138, EGP-2, EGP-40, EpCAM, Erb-B2, Erb-B3, Erb-B4, FBP, Fetal acetylcholine receptor, folate receptor-a, GD2, GD3, HER-2, hTERT, IL-13R-α2, κ-light chain, KDR, mutant KRAS (including, but not limited to, G12V, G12D, G12C), mutant HRAS, mutant PIK3CA (including, but not limited to, E52K, E545K, H1047R, H1047L), mutant IDH (including, but not limited to, R132H), mutant p53 (including, but not limited to, R175H, Y220C, G245D, G245S, R248L, R248Q, R248W, R249S, R273C, R273L, R273H and R282W), mutant NRAS (including, but not limited to, Q61R, Q61K, and Q61L), LeY, L1 cell adhesion molecule, MAGE-A1, Mesothelin, ERBB2, MAGEA3, CT83 (also known as KK-LC-1), p53, MART1,GP100, Proteinase3 (PR1), Tyrosinase, Survivin, hTERT, EphA2, NKG2D ligands, NY-ESO-1, oncofetal antigen (h5T4), PSCA, PSMA, ROR1, TAG-72, VEGF-R2, WT-1, BCMA, CD123, CD44V6, NKCS1, EGF1R, EGFR-VIII, CD99, CD70, ADGRE2, CCR1, LILRB2, PRAIVIE, HPV E6 oncoprotein, HPV E7 oncoprotein, and ERBB. In certain embodiments, the tumor antigen is CD19.

In certain embodiments, the antigen-recognizing receptor binds to a human CD19 polypeptide. In certain embodiments, the antigen-recognizing receptor binds to the extracellular domain of a human CD19 protein.

### 5.4.2 T-Cell Receptor (TCR)

In certain embodiments, the antigen-recognizing receptor is a TCR. A TCR is a disulfide-linked heterodimeric protein consisting of two variable chains expressed as part of a complex with the invariant CD3 chain molecules. A TCR is found on the surface of T cells, and is responsible for recognizing antigens as peptides bound to major histocompatibility complex (MHC) molecules. In certain embodiments, a TCR comprises an alpha chain and a beta chain (encoded by TRA and TRB, respectively). In certain embodiments, a TCR comprises a gamma chain and a delta chain (encoded by TRG and TRD, respectively).

Each chain of a TCR is composed of two extracellular domains comprising a Variable (V) region and a Constant (C) region. The Constant region is proximal to the cell membrane, followed by a transmembrane region and a short cytoplasmic tail that lacks the ability to transduce a signal. The Variable region binds to the peptide/MHC complex. The variable domain of each pair (alpha/beta or gamma/delta) of TCR polypeptides comprises three complementarity determining regions (CDRs).

In certain embodiments, a TCR can form a receptor complex with three dimeric signaling modules CD3δ/ϵ, CD3γ/ϵ and CD3ζ/ζ or ζ/η. When a TCR complex engages with its antigen and MHC (peptide/MHC), the T cell expressing the TCR complex is activated.

In certain embodiments, the TCR recognizes a tumor antigen (including a TAA or TSA). In certain embodiments, the TCR is expressed in a tumor-specific T cell. In certain embodiments, the tumor-specific T cell is a tumor-infiltrating T cell generated by culturing T cells with explants of a tumor, e.g., melanoma or an epithelial cancer. In certain embodiments, the tumor-specific T cell is a T cell disclosed in Stevanovic et al, Science, 356, 200-205, 2017; Dudley et al. Journal of Immunotherapy, 26 (4): 332-342, 2003; or Goff et al, Journal of Clinical Oncology, Vol. 34, No. 20, 2016, each of which is incorporated by reference in its entirety.

In certain embodiments, the antigen-recognizing receptor is a recombinant TCR. In certain embodiments, the recombinant TCR differs from any naturally occurring TCR by at least one amino acid residue. In certain embodiments, the recombinant TCR differs from any naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about about 90, about 100 or more amino acid residues. In certain embodiments, the recombinant TCR is modified from a naturally occurring TCR by at least one amino acid residue. In certain embodiments, the recombinant TCR is modified from a naturally occurring TCR by at least about 2, about 3, about 4, about about 6, about 7, about 8, about 9, about 10, about 11, about 12, about 13, about 14, about 15, about 20, about 25, about 30, about 40, about 50, about 60, about 70, about 80, about 90, about 100 or more amino acid residues.

### 5.4.3 Chimeric Antigen Receptor (CAR)

In certain embodiments, the antigen-recognizing receptor is a CAR. CARs are engineered receptors, which graft or confer a specificity of interest onto an immune effector cell or immunoresponsive cell. CARs can be used to confer non-MHC-restricted antigen specificity onto a T cell. Transfer of their coding sequence facilitated by retroviral vectors.

CARs have developed via a series of significant improvements referred to as “generations”. So-called “first generation” CARs are typically composed of an extracellular antigen-binding domain (e.g., a single chain variable fragment (scFv)), which is fused to a transmembrane domain, which is fused to a cytoplasmic/intracellular signaling domain. The cytoplasmic/intracellular signaling domain can comprise a single activating domain—usually an ITAM derived from CD3zeta. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs add an intracellular signaling domain derived from any one of the various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (CD3ζ). “Third generation” CARs comprise those that provide multiple co-stimulation (e.g., CD28 and 4-1BB) and activation (CD3ζ). In certain embodiments, the antigen-recognizing receptor is a first-generation CAR. In certain embodiments, the antigen-recognizing receptor is a second-generation CAR. In certain embodiments, the antigen-recognizing receptor is a third-generation CAR.

In accordance with the presently disclosed subject matter, a CAR comprises an extracellular antigen-binding domain, a transmembrane domain and an intracellular signaling domain, wherein the extracellular antigen-binding domain specifically binds to an antigen, which can be a tumor antigen (TAA or TSA).

In certain embodiments, the CAR comprises an extracellular antigen-binding domain that binds to CD19. In certain embodiments, the CAR is CTL019 (also known as tisagenlecleucel).

### 5.4.3.1 Extracellular Antigen-Binding Domain of A CAR

In certain embodiments, the extracellular antigen-binding domain specifically binds to an antigen. In certain embodiments, the antigen is a tumor antigen. In certain embodiments, the tumor antigen is a tumor specific antigen (TSA). In certain embodiments, the tumor antigen is a tumor-associated antigen (TAA). In certain embodiments, the tumor antigen is CD19.

In certain embodiments, the extracellular antigen-binding domain is an scFv. In certain embodiments, the scFv is a human scFv. In certain embodiments, the scFv is a humanized scFv. In certain embodiments, the scFv is a murine scFv. In certain embodiments, the extracellular antigen-binding domain is a Fab, which is optionally crosslinked. In certain embodiments, the extracellular antigen-binding domain is a F(ab)₂. In certain embodiments, any of the foregoing molecules may be comprised in a fusion protein with a heterologous sequence to form the extracellular antigen-binding domain. In certain embodiments, the scFv is identified by screening scFv phage library with an antigen-Fc fusion protein.

### 5.4.3.2. Transmembrane Domain of a CAR

In certain embodiments, the transmembrane domain of the CAR comprises a hydrophobic alpha helix that spans at least a portion of the membrane. Different transmembrane domains result in different receptor stability. After antigen recognition, receptors cluster and a signal are transduced to the cell. In accordance with the presently disclosed subject matter, the transmembrane domain of the CAR can comprise a CD8 polypeptide (e.g., a transmembrane domain of CD8), a CD28 polypeptide (e.g., a transmembrane domain of CD28), a CD3ζ polypeptide (e.g., a transmembrane domain of CD3ζ), a CD4 polypeptide (e.g., a transmembrane domain of CD4), a 4-1BB polypeptide (e.g., a transmembrane domain of 4-1BB), an OX40 polypeptide (e.g., a transmembrane domain of OX40), an ICOS polypeptide (e.g., a transmembrane domain of ICOS), a synthetic peptide, or a combination thereof. In certain embodiments, the transmembrane domain of the CAR comprises a CD8 polypeptide (e.g., a transmembrane domain of CD8).

In certain embodiments, the CAR further comprises a spacer region that links the extracellular antigen-binding domain to the transmembrane domain. The spacer region can be flexible enough to allow the antigen binding domain to orient in different directions to facilitate antigen recognition. The spacer region can be the hinge region from IgG1, or the CH₂CH₃ region of immunoglobulin and portions of CD3, a portion of a CD28 polypeptide, a portion of a CD8 polypeptide, or a synthetic spacer sequence. In certain embodiments, the spacer region is derived from a CD8 polypeptide.

### 5.4.3.3 Intracellular Signaling Domain of a CAR

In certain embodiments, the intracellular signaling domain of the CAR comprises a CD3ζ polypeptide, which can activate or stimulate a cell (e.g., a cell of the lymphoid lineage, e.g., a T cell). Wild type (“native”) CD3ζ comprises three immunoreceptor tyrosine-based activation motifs (“ITAMs”) (e.g., ITAM1, ITAM2 and ITAM3), and transduces an activation signal to the cell (e.g., a cell of the lymphoid lineage, e.g., a T cell) after antigen is bound. The intracellular signaling domain of the native CD3ζ polypeptide is the primary transmitter of signals from endogenous TCRs.

In certain embodiments, the intracellular signaling domain of the CAR comprises a native CD3ζ polypeptide. In certain embodiments, the intracellular signaling domain of the CAR comprises a human CD3ζ polypeptide. In certain embodiments, the intracellular signaling domain of the CAR comprises a murine CD3ζ polypeptide. In certain embodiments, the intracellular signaling domain of the CAR comprises a modified CD3ζ polypeptide. In certain embodiments, the intracellular signaling domain of the CAR comprises a modified human or modified murine CD3ζ polypeptide.

In certain embodiments, the intracellular signaling domain of the CAR does not comprise a co-stimulatory signaling region, i.e., the CAR is a first-generation CAR.

In certain embodiments, the intracellular signaling domain of the CAR further comprises at least one co-stimulatory signaling region. In certain embodiments, the co-stimulatory signaling region comprises an intracellular domain of a co-stimulatory molecule or a portion thereof. As used herein, “co-stimulatory molecules” refer to cell surface molecules other than antigen receptors or their ligands that are required for an efficient response of lymphocytes to antigen. Co-stimulatory molecules can provide optimal lymphocyte activation. In certain embodiments, the at least one co-stimulatory signaling region comprises a CD28 polypeptide (e.g., an intracellular domain of CD28 or a portion thereof), a 4-1BB polypeptide (e.g., an intracellular domain of 4-1BB or a portion thereof), an OX40 polypeptide (e.g., an intracellular domain of OX40 or a portion thereof), an ICOS polypeptide (e.g., an intracellular domain of ICOS or a portion thereof), a DAP-10 polypeptide (e.g., an intracellular domain of DAP-10 or a portion thereof), or a combination thereof In certain embodiments, the at least one co-stimulatory signaling region comprises a CD28 polypeptide (e.g., an intracellular domain of CD28 or a portion thereof). In certain embodiments, the at least one co-stimulatory signaling region comprises a 4-1BB polypeptide (e.g., an intracellular domain of 4-1BB or a portion thereof). The co-stimulatory molecule can bind to a co-stimulatory ligand, which is a protein expressed on cell surface that upon binding to its receptor produces a co-stimulatory response, i.e., an intracellular response that effects the stimulation provided when an antigen binds to its CAR molecule. Co-stimulatory ligands, include, but are not limited to CD80, CD86, CD70, OX40L, and 4-1BBL. As one example, a 4-1BB ligand (i.e., 4-1BBL) may bind to 4-1BB (also known as “CD137”) for providing an intracellular signal that in combination with a CAR signal induces an effector cell function of the CAR⁺ T cell.

### 5.4.4. Cells

In certain embodiments, the cell that comprises an antigen-recognizing receptor is an immunoresponsive cell. In certain embodiments, the cell is a cell of the lymphoid lineage. Cells of the lymphoid lineage produce antibodies, regulate cellular immune system, and detect foreign agents in the blood and cells foreign to the host and the like. Non-limiting examples of cells of the lymphoid lineage include T cells, Natural Killer (NK) cells, B cells, dendritic cells, and stem cells from which lymphoid cells may be differentiated. In certain embodiments, the stem cell is a pluripotent stem cell (e.g., embryonic stem cell or induced pluripotent stem cell). In certain embodiments, the cell is an antigen presenting cell.

In certain embodiments, the cell is a T cell. T cells can be lymphocytes that mature in the thymus and are chiefly responsible for cell-mediated immunity. T cells are part of the adaptive immune system. In certain embodiments, the T cells provided herein comprise any type of T cells, including, but not limited to, helper T cells, cytotoxic T cells, memory T cells (including central memory T cells, stem-cell-like memory T cells (or stem-like memory T cells), and two types of effector memory T cells: e.g., T_{EM} cells and T_{EMRA} cells, regulatory T cells (also known as suppressor T cells or T_{regs}), tumor-infiltrating lymphocytes (TILs), natural killer T cells, mucosal associated invariant T cells, and γδ T cells. Cytotoxic T cells (CTLs or killer T cells) are a subset of T lymphocytes capable of inducing the death of infected somatic or tumor cells. A patient's own T cells (i.e., autologous T cells) may be genetically modified to target specific antigens through the introduction of an antigen-recognizing receptor, e.g., a CAR or a TCR. In certain embodiments, the cell is a T cell. The T cell can be a CD4⁺ T cell or a CD8⁺ T cell. In certain embodiments, the T cell is a CD4⁺ T cell. In certain embodiments, the T cell is a CD8⁺ T cell.

In certain embodiments, the cell is a tumor-specific T cell. In certain embodiments, the tumor-specific T cell comprises an endogenous TCR that recognizes a tumor antigen (TSA or TAA).

In certain embodiments, the cell is an NK cell. Natural killer (NK) cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes, e.g., those disclosed in Sadelain, M., et al. 2003 Nat Rev Cancer 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli, M. C., et al. 2000 J Immunol 164:495-504; Panelli, M. C., et al. 2000 J Immunol 164:4382-4392 (disclosing lymphocyte cultures derived from tumor infiltrating lymphocytes (TILs) in tumor biopsies), and in Dupont, J., et al. 2005 Cancer Res 65:5417-5427; Papanicolaou, G. A., et al. 2003 Blood 102:2498-2505 (disclosing selectively in vitro-expanded antigen-specific peripheral blood leukocytes employing artificial antigen-presenting cells (AAPCs) or pulsed dendritic cells). The immunoresponsive cells (e.g., T cells) can be autologous, non-autologous (e.g., allogeneic), or derived in vitro from engineered progenitor or stem cells.

In certain embodiments, the cell is a cell of the myeloid lineage. Non-limiting examples of cells of the myeloid lineage include monocytes, macrophages, basophils, neutrophils, eosinophils, mast cell, erythrocytes, megakaryocytes, thrombocytes, and stem cells from which myeloid cells may be differentiated. In certain embodiments, the stem cell is a pluripotent stem cell (e.g., embryonic stem cell or induced pluripotent stem cell).

### 5.5 Administration

In certain embodiments, the personalized amount of the chemotherapeutic agent can be administered by parenteral, subcutaneous, intramuscular, intravenous, intrarticular, intrabronchial, intraabdominal, intracapsular, intracartilaginous, intracavitary, intracelial, intracerebellar, intracerebroventricular, intrathecal, intra-Ommaya, intraocular, intravitreous, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, or transdermal routes. In certain embodiments, the methods disclosed herein comprise administer the personalized amount of chemotherapeutic agent intravenously (IV).

The chemotherapeutic agent can be administered in a single dose, or in multiple doses. In certain embodiments, the chemotherapeutic agent is administered to the subject in multiple doses. The multiple doses can be administered sequentially, e.g., at daily, weekly, or monthly intervals, or in response to a specific need of the subject. In certain embodiments, the chemotherapeutic agent is administered to the subject daily in 2, 3, 4, or 5 consecutive days.

In certain embodiments, the cells comprising the antigen-recognizing receptor are administered to the subject by directly injecting into an organ of interest (e.g., an organ affected by a tumor). In certain embodiments, the cells comprising the antigen-recognizing receptor are administered to the subject by indirectly injecting to organ of interest, for example, by administration into the circulatory system (e.g., the tumor vasculature).

In certain embodiments, the cells can be administered in any physiologically acceptable vehicle, normally intravascularly, although they may also be introduced into bone or other convenient site where the cells may find an appropriate site for regeneration and differentiation (e.g., the thymus). Usually, at least about 1×10⁵ cells will be administered, eventually reaching about 1×10¹⁰ or more. In certain embodiments, the cells can comprise a purified population of cells. Those skilled in the art can readily determine the percentage of the presently disclosed cells in a population using various well-known methods, such as fluorescence activated cell sorting (FACS). The cells can be introduced by injection, catheter, or the like.

In certain embodiments, the cells can be autologous or heterologous. For example, cells or progenitors can be obtained from one subject, and administered to the same subject or a different, compatible subject. Peripheral blood derived cells or their progeny (e.g., in vivo, ex vivo or in vitro derived) can be administered via localized injection, including catheter administration, systemic injection, localized injection, intravenous injection, or parenteral administration.

### 6. EXAMPLES

The present disclosure will be better understood by reference to the following Example, which is provided as exemplary of the present disclosure, and not by way of limitation.

### Example 1

### Fludarabine-Exposure Predicts Disease Control Following CD19-Specific CAR T Cell (Tisagenleucleucel)

A retrospective analysis was performed on data abstracted from the Pediatric Real-World CAR Consortium (PRWCC) consisting 15 different US institutions. Patients who received standard fludarabine dosing (30 mg/m²/daily×4 days) and had ≤3 days between chemotherapy and infusion were included. The area under the curve (AUC) of fludarabine exposure was calculated using a previously published population-PK model (Langenhorst et al., Blood Advances (2019);3:2179-2187). Main outcomes of interest included: response rate, relapse-free (RFS) and overall survival (OS), a composite endpoint of loss of B-cell aplasia (BCA) and/or relapse.

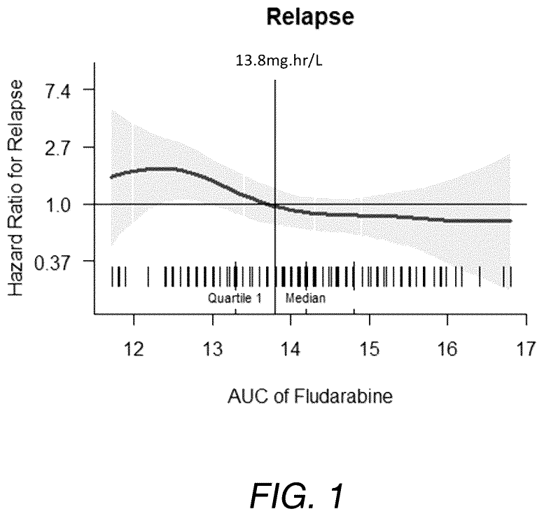

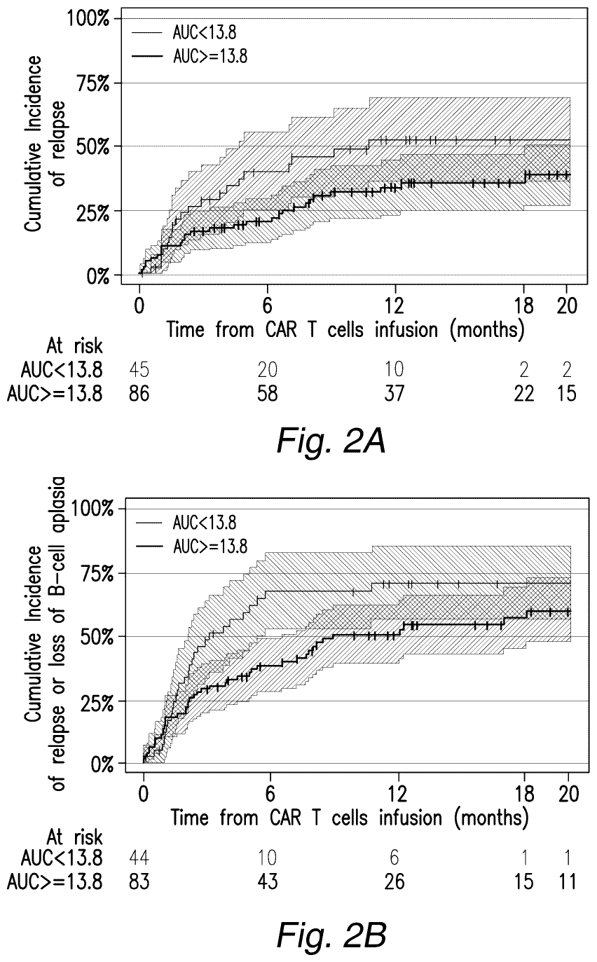

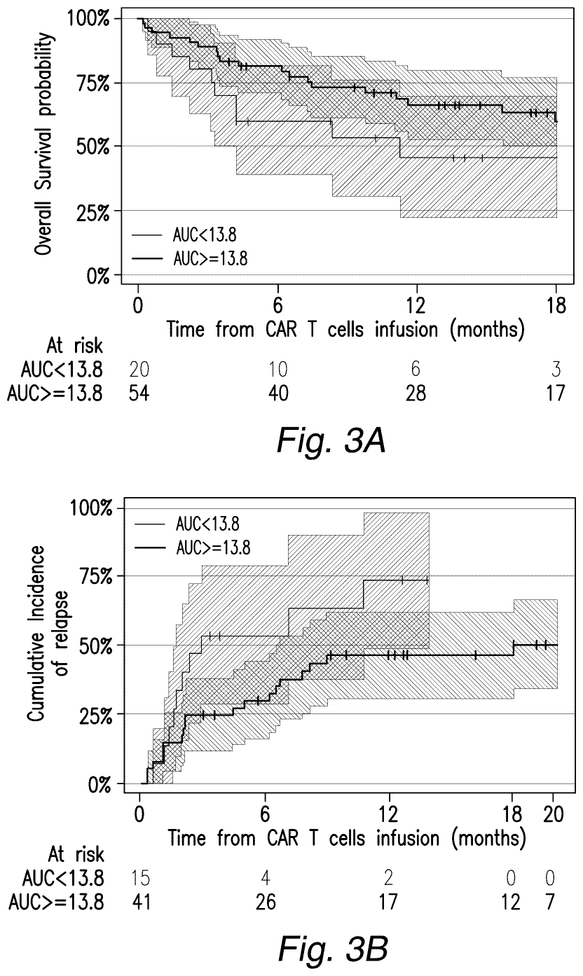

Total 152 patients were included in the study. The median age was 13 years old (range between <1 and 26 years old). The estimated median fludarabine AUC prior to tisagenlecleucel (CD19-specific CAR T cell) infusion was 14.4 mg·hr/L (range between 11.1 and 22.4). Complete remission was achieved in 86% (n=131) of patients, the 12-month cumulative incidence of relapse in responders was 39.2% (95% CI: 30.2-48.2%), and the 12-month OS was 75% (95% CI: 67.6-82.6%). Fludarabine exposure was analyzed as a continuous variable. An AUC of 13.8 mg·hr/L was demonstrated to be a threshold of interest (FIG. 1) for disease relapse. Patients who received a fludarabine AUC of ≥13.8 mg·hr/L had a lower risk of relapse (p=0.03; FIG. 2A) and loss of BCA/relapse ((composite endpoint) p-0.03; FIG. 2B). The PRWCC previously reported pre-treatment disease burden negatively impacts OS and disease control (Schultz et al., Blood (2020) 136:14-15). Analysis of this sub-cohort of patients with high pre-treatment disease burden demonstrated that fludarabine AUC ≥13.8mg·hr/L was associated with improved OS (p=0.03) and a lower incidence of relapse (p=0.03; FIGS. 3A-3B).

Fludarabine-exposure impacted outcomes after CAR-T cell treatment. Higher fludarabine exposure (AUC≥13.8 mg·hr/L) was associated with lower relapse rates and protected BCA regardless of pre-treatment disease burden. Higher fludarabine exposure was also associated with higher OS. Also, in patients with high-disease burden, optimizing fludarabine exposure by personalized dosing can improve the durability of CD19-specific CAR T cell therapy and subsequent survival.

### Example 2

### Fludarabine Lymphodepletion is Associated With Improved Outcomes Following CAR T Cell Therapy

Acute lymphoblastic leukemia (ALL) is the most common malignancy occurring in children, and for patients with relapsed or refractory (R/R) B-cell ALL (B-ALL) the prognosis is dismal (Nguyen et al., Leukemia. December 2008; 22 (12):2142-50; Gaynon P S, Br J Haematol. December 2005; 131 (5):579-87; and von Stackelberg et al., Eur J Cancer. January 2011; 47 (1):90-7). It has been reported that the use of CD19-specific CAR T cells as a therapeutic option for patients with R/R B-ALL (Davila et al., Sci Transl Med. February 2014; 6 (224):224ra25; Maude et al., N Engl J Med. October 2014; 371 (16):1507-17; Lee et al., Lancet. February 2015; 385 (9967):517-528; Maude, Clin Adv Hematol Oncol. October 2018; 16 (10):664-666; Gardner et al., Blood. 06 2017; 129 (25):3322-3331; Curran et al., Blood. Oct. 17, 2019; Park et al., N Engl J Med. 02 2018; 378 (5):449-459; Frey et al., J Clin Oncol. Dec. 9 2019; and Maude et al., N Engl J Med. 2018; 378 (5):439-448). Accessibility of this cellular therapy was broadened with the U.S. Food and Drug Administration's approval of tisagenlecleucel, a CD19-specific CAR T cell for R/R B-ALL in patients <26 years old (Maude et al., N Engl J Med. 2018; 378 (5):439-448). Despite initial response to CD19-specific CAR T cell therapy in the setting of R/R B-ALL, the incidence of relapse among responders is estimated at 40-50%, demonstrating a need to improve this treatment strategy (Maude et al., N Engl J Med. October 2014; 371 (16):1507-17; Lee et al., Lancet. February 2015; 385 (9967):517-528; Maude, Clin Adv Hematol Oncol. October 2018; 16 (10):664-666; and Park et al., N Engl J Med. 02 2018; 378 (5):449-459). The current successful application of CAR T cell therapy has required pre-treatment of patients with lymphodepleting chemotherapy (LDC). Most commonly LDC utilizes a combination of cyclophosphamide and fludarabine; however, dosing, duration, and intensity of LDC have varied across institutions and trials (Davila et al., Sci Transl Med. February 2014; 6 (224):224ra25; Maude et al., N Engl J Med. October 2014; 371 (16):1507-17; Lee et al., Lancet. February 2015; 385 (9967):517-528; Maude, Clin Adv Hematol Oncol. October 2018; 16 (10):664-666; Gardner et al., Blood. 06 2017; 129 (25):3322-3331; Curran et al., Blood. Oct. 17, 2019; Park et al., N Engl J Med. 02 2018; 378 (5):449-459; Frey et al., J Clin Oncol. Dec. 9, 2019; and Maude et al., N Engl J Med. 2018; 378 (5):439-448). The addition of fludarabine to cyclophosphamide has been shown to improve CAR T cell kinetics, initial response, and decrease rejection in both B-ALL and non-Hodgkin lymphoma (NHL) (Gardner et al., Blood. 06 2017; 129 (25):3322-3331; Curran et al., Blood. Oct. 17, 2019; Turtle et al., Sci Transl Med. Sep. 7, 2016; 8 (355):355ra1 16; and Turtle et al., J Clin Invest. 06 2016; 126 (6):2123-38). Dose intensity of cyclophosphamide has also been shown to improve response and peak CAR T cell expansion in pediatric and young adults with R/R B-ALL and progression free survival and cytokine milieu in adult patients with R/R NHL (Curran et al., Blood. Oct. 17, 2019; Hirayama et al., Blood. Apr. 25, 2019; 133(17):1876-1887). In patients undergoing allogeneic hematopoietic cell transplantation (allo-HCT), optimal fludarabine exposure has been found to decrease non-relapse mortality due to improved immune reconstitution and subsequently improve survival (Langenhorst et al., Blood Adv. Jul. 23, 2019). Fludarabine exposure has not yet been studied in patients undergoing CAR T cell therapy. The presently disclosed subject matter reports the results of a cohort of pediatric and young adult patients with R/R B-ALL who received tisagenlecleucel and defines the optimal fludarabine exposure associated with improved outcomes.

### Methods

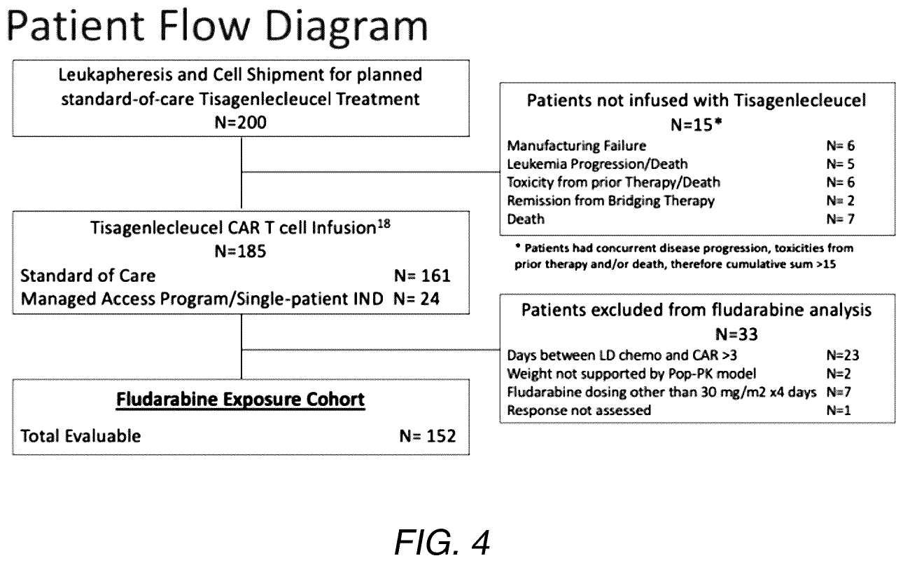

Study Design. A retrospective analysis of pediatric and young adult patients with R/R B-ALL as part of the Pediatric Real-World CAR Consortium (PRWCC; n=15 centers) was conducted. Two hundred (n=200) patients underwent collection/manufacture for planned standard-of-care CD19-specific CAR T cell therapy (tisagenlecleucel). Patients in this analysis were excluded if they did not receive their tisagenlecleucel infusion (n=15), they did not have evaluable response data at the cutoff date (n=1), they received lymphodepletion other than the tisagenlecleucel package insert LDC (fludarabine 30 mg/m²/day×4 days and cyclophosphamide 500 mg/m²/day×2 days; n=7), their weight was not supported by the population pharmacokinetic (PK) model (n=2), or the number of days between lymphodepletion and infusion was >3 days (n=23; FIG. 4).

Outcomes of interest. The main outcomes of interest were overall survival (OS), cumulative incidence of relapse (CIR), and the cumulative incidence of a composite endpoint (relapse or loss of BCA). Other outcomes of interest were response rates, cytokine release syndrome (CRS), and immune effector cell associated neurotoxicity syndrome (ICANS). Complete remission (CR) was defined as ≤5% bone marrow blasts by morphology, absence of circulating lymphoblasts, and no evidence of extramedullary disease. MRD negativity was defined as <0.01% abnormal B cells assessed by flow cytometry. Relapse was defined as any (medullary or extramedullary) evidence of hematologic, cytogenetic, and/or molecular recurrence of primary disease. CD19-positive B cell recovery was defined as any detectable CD19-positive B cells (>1 cell/mL) on a peripheral blood lymphocyte flow cytometry panel. Toxicity grading was described according to ASTCT (Lee et al., Biol Blood Marrow Transplant. 04 2019; 25 (4):625-638) for CRS and ICANS. Additionally, patients were further delineated based on their disease burden: high disease burden (≥5% lymphoblasts, CNS3 disease and/or isolated EM disease), and no (undetectable) or low disease burden (<5% lymphoblasts, ≤CNS2 disease, and/or no detectable EM disease) based off the previous PRWCC analysis (Schultz et al., Blood. 2020; 136 (Supplement 1):14-15). The fludarabine exposure, area under the curve (AUC), for each patient was calculated using a population PK model as previously described in Langenhorst et al., Clin Pharmacokinet. May 2019; 58 (5):627-637. The patient variables required to calculate an estimated fludarabine exposure include estimated GFR (using either the Cockroft-Gault or Schwartz equation depending on age), actual body weight, height, and dosage of fludarabine utilized where actual body weight and estimated GFR are the best predictors of fludarabine PK (Langenhorst et al., Clin Pharmacokinet. May 2019; 58 (5):627-637).

Statistical Analysis. For all analyses, tisagenlecleucel infusion date was considered time zero (0). The impact of different factors on the chance of response was assessed through Mann-Whitney-Wilcoxon test for continuous variables, Chi-squared or Fisher's exact test for categorical variables (univariable analysis), and logistic regression (multivariable analysis). OS for all treated patients was defined as the time from infusion to death from all causes. OS in responding patients was defined as time from day of response (day 28 from CAR infusion) to death from all causes. Patients alive were censored at their last follow-up date. Time-to-relapse was defined as the time from the date of infusion to the date of disease relapse, while time-to-composite endpoint was defined as the time from the date of infusion to the date of either disease relapse or loss of BCA, whichever came first. For both endpoints, patients alive without disease relapse (and loss of BCA for composite endpoint) were censored at their date of last follow-up. Death and allo-HCT without disease relapse (and loss of BCA) were considered competing events. Patients who died prior to day 28 or did not achieve a CR after tisagenlecleucel were not at risk of relapse and therefore were excluded from the cumulative incidence of relapse and composite endpoint analyses. Kaplan-Meier analysis was used to estimate OS while an Aalen-Johansen estimator was used for the cumulative incidence of relapse and the composite endpoint (prodlim: Product-Limit Estimation for Censored Event History Analysis. Version 2018.04.18. CRAN.R-project.org. The potential follow-up was estimated using reverse Kaplan-Meier estimate. Differences in survival curves between groups were tested using a log-rank test or Gray's test for cumulative incidences. The impact of clinical factors associated with response and fludarabine exposure on survival endpoints was assessed using Cox models (cause-specific hazard models in presence of competing risks). The estimated fludarabine (cumulative) AUC for each patient was plotted as a continuous variable against hazard ratios for relapse and optimal exposure was explored using Hazard Ratio plots (Greg: Regression Helper Functions. Version 1.3.1. 2019. https://CRAN.R-project.org/package=Greg). In both logistic and Cox models, variables with p-value <0.1 in univariable analysis were included in multivariable analysis. A p-value <0.05 was considered statistically significant.

### Results

Patient Characteristics. One-hundred fifty-two (n=152) patients met inclusion criteria for analysis (FIG. 4). Baseline patient characteristics are shown in Table 1.

TABLE 1
Patient characteristics and outcomes.

| Patient characteristics | n = 152 | |
|---|---|---|
| Median age at infusion (years; (range)) | 12.5 | (<1-26) |
| Median age at diagnosis (years; (range)) | 8 | (<1-25) |
| Gender (male/female) | 93/59 | |
| Race | | |
| Asian | 6 | (4%) |
| Black | 7 | (5%) |
| Hispanic | 58 | (39%) |
| More than 1 race | 2 | (1%) |
| White | 76 | (51%) |
| Unknown | 3 | |
| Initial cytogenetic risk | | |
| Favorable | 22 | (19%) |
| Intermediate | 42 | (36%) |
| Unfavorable | 53 | (45%) |
| Unknown | 35 | |
| Pre-infusion disease burden | | |
| No detectable disease | 41 | (28%) |
| Low disease | 33 | (22%) |
| High disease | 74 | (50%) |
| Unknown | 4 | |
| Mean time diagnosis to infusion (months) | 43 | (3-164) |
| Mean number of prior lines of therapy | 3.5 | (1-10) |
| Prior allo-HCT | 8 | (5%) |
| Prior CD19 directed therapy | 33 | (22%) |
| Disease status | | |
| Refractory disease | 24 | (16%) |
| ≥1 relapse | 128 | (84%) |
| Overall day 28 response | | |
| No CR | 16 | (11%) |
| CR | 131 | (86%) |
| Died prior to day28 | 5 | (3%) |
| CRS grade | | |
| none | 55 | (36%) |
| 1 | 36 | (24%) |
| 2 | 28 | (18%) |
| 3 | 17 | (11%) |
| 4 | 15 | (10%) |
| 5 | 1 | (1%) |
| Unknown | 1 | |
| Neurotoxicity grade | | |
| none | 116 | (76%) |
| 1 | 17 | (11%) |
| 2 | 7 | (5%) |
| 3 | 8 | (5%) |
| 4 | 3 | (2%) |
| 5 | 1 | (1%) |
| Relapsed post CAR | 55 | (36%) |
| CD19-positive relapse | 28 | (55%) |
| CD19-negative relapse | 23 | (45%) |
| unknown | 4 | |
| Loss of BCA post CAR | 52 | (34%) |
| Survival status (alive/dead) | 112/40 | |
| Cause of death | | |
| Leukemia | 30 | (74%) |
| Infection | 4 | (10%) |
| CRS | 1 | (2%) |
| Neurotoxicity | 1 | (2%) |
| Transplant related | 3 | (7%) |
| ARDS/cardiac arrest | 1 | (2%) |

allo-HCT, allogeneic hematopoietic cell transplantation; CR, complete remission; CRS, cytokine release syndrome; CAR, chimeric antigen receptor; BCA, B cell aplasia; ARDS, acute respiratory distress syndrome.

The median age at diagnosis was 8 years (<1-25 years) and the median age at tisagenlecleucel infusion was 12.5 years (range <1-26 years). The median time from initial diagnosis to infusion was 2.9 years (range 0.3-13.7). Complete remission was achieved in 86% (131/152) of patients. Death prior to day 28 disease assessment occurred in 3% (5/152) of patients and 11% (16/152) of patients did not achieve CR. Univariable analysis of baseline patient characteristics found pre-infusion disease burden (p=0.001), prior CD19 directed therapy (p=0.01), and race (p=0.025) as factors associated with response (Table 2). The PRWCC previously reported that pre-infusion disease burden and race were prognostic for response (Schultz et al., Blood. 2020; 136 (Supplement 1):14-15; Baggott et al., 2021 TCT Transplantation & Cellular Therapy Meetings of ASTCT and CIBMTR. TCT Meetings, 2021).

TABLE 2
Univariable analysis of disease characteristics and response.

| | Non-responders | | Responders | | |
|---|---|---|---|---|---|
| Characteristics | n = 16 | | n = 131 | | p-value |
| Age at diagnosis | | | | | >0.99 |
| <1 year or >10 years | 8 | (12%) | 61 | (88%) | |
| 1 to 10 years | 8 | (10%) | 70 | (90%) | |
| Gender (male/female) | 10/6 | | 81/50 | | >0.99 |
| Race | | | | | 0.025 |
| Black/Hispanic | 12 | (19%) | 50 | (81%) | |
| Others | 0 | (0%) | 8 | (100%) | |
| White | 4 | (5%) | 70 | (95%) | |
| Unknown | 0 | | 3 | | |
| Initial cytogenetic risk | | | | | 0.44 |
| Favorable | 4 | (18%) | 18 | (82%) | |
| Intermediate | 4 | (10%) | 37 | (90%) | |
| Unfavorable | 4 | (8%) | 46 | (92%) | |
| Unknown | 4 | | 30 | | |
| Pre-infusion disease burden | | | | | 0.001 |
| No or low disease | 1 | (1%) | 73 | (99%) | |
| High disease | 13 | (19%) | 56 | (81%) | |
| Unknown | 2 | | 2 | | |
| Time diagnosis to infusion | 36 | (6-117) | 45 | (3-164) | 0.44 |
| (months) | | | | | |
| Number of prior lines | 3.9 | (2-7) | 3.4 | (1-10) | 0.14 |
| of therapy | | | | | |
| Prior allo-HCT | | | | | 0.56 |
| no | 15 | (11%) | 125 | (89%) | |
| yes | 1 | (14%) | 6 | (86%) | |
| Prior CD19 directed therapy | | | | | 0.01 |
| no | 8 | (7%) | 106 | (93%) | |
| yes | 8 | (24%) | 25 | (76%) | |
| Disease status | | | | | >0.99 |
| Refractory disease | 2 | (8%) | 22 | (92%) | |
| ≥1 relapse | 14 | (11%) | 109 | (89%) | |

allo-HCT, allogeneic hematopoietic cell transplantation

Fludarabine Exposure and Response. The mean dose of fludarabine received was 41 mg per dose (range 11-70 mg) and the estimated mean fludarabine cumulative AUC prior to tisagenlecleucel infusion was 14.4 mg*hr/L (sd=1.6, range 11.1-22.4 mg*hr/L). The impact of fludarabine AUC on response was explored in the entire cohort. In context of a limited sample of non-responding patients (n=16), fludarabine AUC was found to be higher in non-responders than responders, with a mean of 14.7 mg*hr/L (sd=1.6, range 11.1-16.7) and 14.4 mg*hr/L (sd=1.5, range 11.7-22.4) respectively (Mann-Whitney-Wilcoxon, p=0.046). Univariable analysis of baseline characteristics and response is shown in Table 2. Multivariable analysis of baseline characteristics prognostic for response in this cohort (race, pre-infusion disease burden, and prior CD19 directed therapy) revealed that fludarabine AUC was not predictive of response after adjustment (p=0.26; Table 3).

TABLE 3
Multivariable analysis of response by patient's characteristics
significant from univariable analysis.

| Characteristics | Odds Ratio | 95% CI | p-value |
|---|---|---|---|
| Cumulative Flu AUC (mg*hr/L) | 0.81 | 0.56-1.21 | 0.26 |
| Race* | | | 0.019 |
| Black/Hispanic | Ref | 1.37-25.7 | |
| White | 5.28 | | |
| Pre-infusion disease burden | Ref | 0.00-0.32 | <0.001 |
| No or low disease | | | |
| High disease | 0.06 | | |
| Prior CD19 directed therapy | | 0.04-0.60 | 0.007 |
| no | Ref | | |
| yes | 0.16 | | |

Flu, fludarabine;
AUC, area under the curve;
ref, reference;
CI, confidence intervals
*No events were observed in the other race groups

Fludarabine Exposure and Outcomes. The median potential follow-up was 1.1 year (Q1=0.8, Q3=1.7 years). Following response, 52 patients relapsed and the 12-month CIR in responding patients was 36.4% (95% CI: 27.5-45.2%). The cumulative incidence of a clinically important composite endpoint (relapse or the loss of BCA) was 55.2% (95% CI: 46.0-64.4) at 12-months with relapse or loss of BCA occurring in 76 patients and only loss of BCA occurring in 52 patients post tisagenlecleucel. A total of 40 patients died in this cohort. The 12-month and 24-month OS for the entire cohort was 75.1% (95% CI: 67.6-82.6%) and 56.5% (95% CI: 41.8-71.2%), respectively.

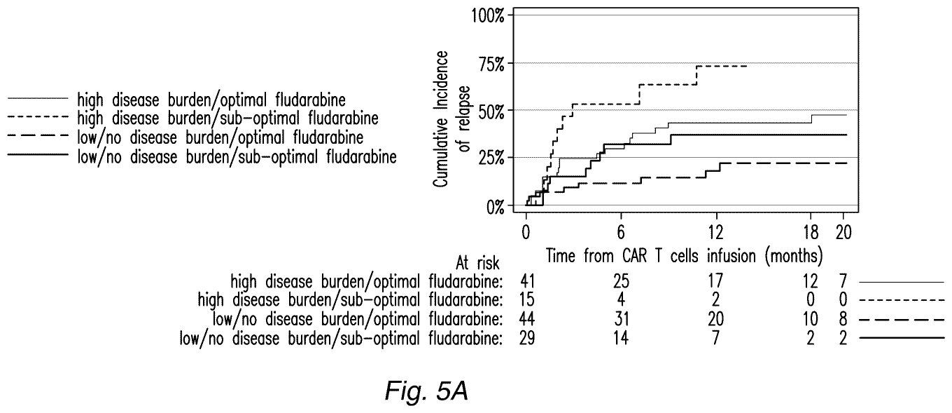

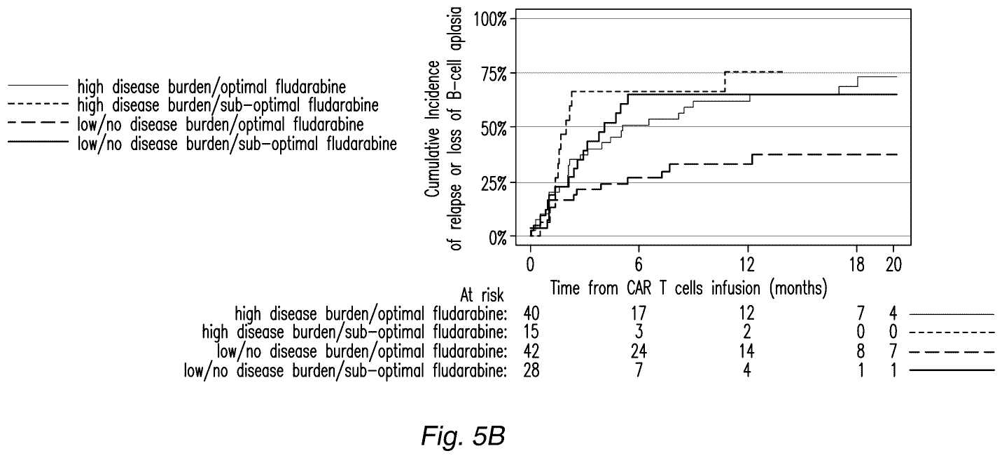

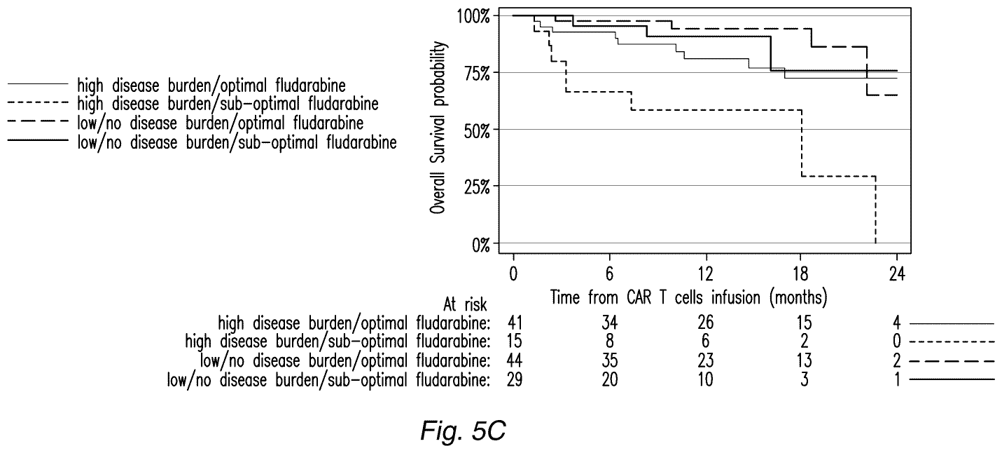

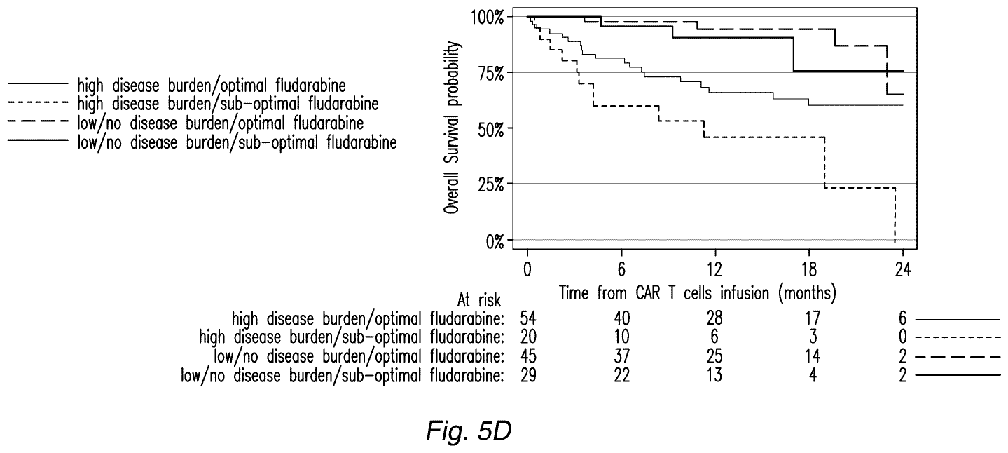

Estimated fludarabine AUC for each patient was plotted as a continuous variable against cumulative risk of relapse (FIG. 1). Optimal fludarabine exposure was defined as an AUC ≥13.8 mg*hr/L as this is where, graphically, the hazard ratio curve for CIR crosses hazard ratio (HR)=1 (FIG. 1). This threshold was utilized as the optimal fludarabine exposure for all outcomes of interest (OS, CIR, and composite endpoint), as the threshold for other endpoints was similar (data not shown). In this cohort, 67% (102/152) had optimal fludarabine exposure. Univariable and multivariable analyses incorporating baseline patient factors and covariate of interest, optimal/sub-optimal fludarabine exposure, were analyzed with outcomes of interest (Tables 4A-4C). After multivariable adjustment, both high pre-infusion disease burden and sub-optimal fludarabine exposure were associated with an increased risk of relapse (HR=2.66; p=0.001 and HR=2.45; p=0.005) and increased composite endpoint (HR=2.02; p=0.005 and HR=1.96; p=0.01; Tables 4A-4C). OS was not associated with sub-optimal fludarabine exposure in the entire cohort (HR=1.53; p=0.26) or when analyzing responding patients only (HR=1.97; p=0.14), whereas pre-infusion disease burden (HR=4.77; p<0.001 and HR=2.90; p=0.013) and age (HR=0.41; p=0.008 and HR=0.34; p=0.010) were associated with survival as previously reported by the PRWCC (Tables 4A-4C and 5; Schultz et al., Blood. 2020; 136 (Supplement 1):14-15). Analysis of patients with high pre-infusion disease burden did associate sub-optimal fludarabine exposure with reduced overall survival for all treated patients (HR=2.27; p=0.03) and in responding patients only (HR=3.50; p=0.02). Outcomes of interest for cohorts of patients based off fludarabine exposure and pre-treatment disease burden are shown in FIGS. 5A-5D. Patients who received optimal or sub-optimal fludarabine exposure had no difference in pre-treatment disease burden or other factors associated with response in this cohort (Table 6).

TABLE 4A
Univariable and multivariable analysis of outcomes of interest based on patient
characteristics in responding patients: cumulative incidence of relapse.

| | Univariable | | | Multivariable | | |
|---|---|---|---|---|---|---|
| Characteristics | HR | 95% CI | p-value | HR | 95% CI | p-value |
| Sex | Ref | | 0.13 | | | |
| Female | 0.64 | 0.36-1.13 | | | | |
| Male | | | | | | |
| Number of prior lines of therapy | 1.09 | 0.92-1.28 | 0.33 | | | |
| Time dx to infusion (months) | 0.99 | 0.99-1.00 | 0.20 | | | |
| Age at diagnosis | | | 0.005 | | | 0.075 |
| <1 or >10 years | Ref | | | Ref | | |
| 1 to 10 years | 0.43 | 0.24-0.78 | | 0.55 | 0.29-1.06 | |
| Pre-infusion disease burden | | | 0.006 | | | 0.001 |
| No or low disease | Ref | | | Ref | | |
| High disease | 2.26 | 1.25-4.10 | | 2.66 | 1.45-4.87 | |
| Initial cytogenetic risk | | | 0.43 | | | |
| Favorable | Ref | | | | | |
| Intermediate | 1.87 | 0.68-5.15 | | | | |
| Unfavorable | 1.68 | 0.62-4.53 | | | | |
| Prior HCT | | | 0.92 | | | |
| No | Ref | | | | | |
| Yes | 0.93 | 0.23-3.85 | | | | |
| Prior CD19 directed therapy | | | 0.43 | | | |
| No | Ref | | | | | |
| Yes | 1.32 | 0.67-2.61 | | | | |
| Prior relapse | | | 0.062 | | | 0.14 |
| 1 or more relapse | Ref | | | Ref | | |
| Refractory | 0.42 | 0.15-1.17 | | 0.49 | 0.18-1.38 | |
| CNS or EM disease | | | 0.17 | | | |
| No | Ref | | | | | |
| Yes | 1.50 | 0.84-2.68 | | | | |
| Race | | | 0.98 | | | |
| Black/Hispanic | Ref | | | | | |
| Others | 1.14 | 0.34-3.89 | | | | |
| White | 1.02 | 0.55-1.88 | | | | |
| Cumulative Flu AUC (mg * hr/L) | | | 0.029 | | | 0.005 |
| ≥13.8 | Ref | | | Ref | | |
| <13.8 | 1.95 | 1.08-3.52 | | 2.45 | 1.34-4.48 | |

TABLE 4B
Univariable and multivariable analysis of outcomes of interest based on patient
characteristics in responding patients: cumulative incidence of composite endpoint (relapse
or loss of B-cell aplasia).

| | Univariable | | | Multivariable | | |
|---|---|---|---|---|---|---|
| Characteristics | HR | 95% CI | p-value | HR | 95% CI | p-value |
| Sex | | | 0.40 | | | |
| Female | Ref | | | | | |
| Male | 0.81 | 0.50-1.31 | | | | |
| Number of prior lines of therapy | 1.04 | 0.90-1.19 | 0.63 | | | |
| Time dx to infusion (months) | 1.00 | 0.99-1.00 | 0.22 | | | |
| Age at diagnosis | | | 0.052 | | | 0.21 |
| <1 or >10 years | Ref | | | Ref | | |
| 1 to 10 years | 0.62 | 0.39-1.00 | | 0.72 | 0.43-1.21 | |
| Pre-infusion disease burden | | | 0.014 | | | 0.005 |
| No or low disease | Ref | | | Ref | | |
| High disease | 1.82 | 1.13-2.95 | | 2.02 | 1.24-3.29 | |
| Initial cytogenetic risk | | | 0.54 | | | |
| Favorable | Ref | | | | | |
| Intermediate | 1.27 | 0.58-2.80 | | | | |
| Unfavorable | 1.51 | 0.71-3.24 | | | | |
| Prior HCT | | | 0.64 | | | |
| No | Ref | | | | | |
| Yes | 0.77 | 0.24-2.44 | | | | |
| Prior CD19 directed therapy | | | 0.82 | | | |
| No | Ref | | | | | |
| Yes | 1.07 | 0.60-1.90 | | | | |
| Prior relapse | | | 0.55 | | | |
| 1 or more relapse | Ref | | | | | |
| Refractory | 1.22 | 0.64-2.33 | | | | |
| CNS or EM disease | | | 0.93 | | | |
| No | Ref | | | | | |
| Yes | 1.02 | 0.64-1.64 | | | | |
| Race | | | 0.71 | | | |
| Black/Hispanic | Ref | | | | | |
| Others | 0.63 | 0.19-2.07 | | | | |
| White | 0.99 | 0.60-1.62 | | | | |
| Cumulative Flu AUC (mg * hr/L) | | | 0.025 | | | 0.010 |
| ≥13.8 | Ref | | | Ref | | |
| <13.8 | 1.77 | 1.09-2.88 | | 1.96 | 1.19-3.23 | |

TABLE 4C
Univariable and multivariable analysis of outcomes of interest based on patient
characteristics in responding patients: overall survival in responding patients

| | Univariable | | | Multivariable | | |
|---|---|---|---|---|---|---|
| Characteristics | HR | 95% CI | p-value | HR | 95% CI | p-value |
| Sex | | | 0.10 | | | 0.69 |
| Female | Ref | | | Ref | | |
| Male | 0.51 | 0.23-1.13 | | 0.83 | 0.35-2.01 | |
| Number of prior lines of therapy | 1.16 | 0.93-1.45 | 0.20 | | | |
| Time dx to infusion (months) | 0.99 | 0.98-1.00 | 0.31 | | | |
| Age at diagnosis | | | 0.011 | | 0.14-0.80 | 0.010 |
| <1 or >10 years | Ref | | | Ref | | |
| 1 to 10 years | 0.34 | 0.14-0.80 | | 0.34 | | |
| Pre-infusion disease burden | Ref | | 0.013 | Ref | | 0.013 |
| No or low disease | | | | | | |
| High disease | 2.88 | 1.19-6.96 | | 2.90 | 1.20-7.00 | |
| Initial cytogenetic risk | | | 0.41 | | | |
| Favorable | Ref | | | | | |
| Intermediate | 2.67 | 0.56-12.7 | | | | |
| Unfavorable | 1.96 | 0.42-9.11 | | | | |
| Prior HCT | | | 0.88 | | | |
| No | Ref | | | | | |
| Yes | 0.86 | 0.12-6.37 | | | | |
| Prior CD19 directed therapy | | | 0.98 | | | |
| No | Ref | | | | | |
| Yes | 1.01 | 0.38-2.71 | | | | |
| Prior relapse | | | 0.19 | | | |
| 1 or more relapse | Ref | | | | | |
| Refractory | 0.42 | 0.10-1.80 | | | | |
| CNS or EM disease | | | 0.68 | | | |
| No | Ref | | | | | |
| Yes | 0.84 | 0.37-1.90 | | | | |
| Race | | | 0.73 | | | |
| Black/Hispanic | Ref | | | | | |
| Others | 1.20 | 0.26-5.44 | | | | |
| White | 0.75 | 0.33-1.73 | | | | |
| Cumulative Flu AUC (mg * hr/L) | | | 0.056 | | | 0.14 |
| ≥13.8 | Ref | | | Ref | | |
| <13.8 | 2.25 | 1.00-5.06 | | 1.97 | 0.80-4.85 | |

HR, hazard ratio;
CI, confidence interval;
dx, diagnosis;
HCT, hematopoietic cell transplantation;
CNS, central nervous system;
EM, extramedullary disease;
flu, fludarabine;
AUC, area under the curve;
ref, reference

TABLE 5
Univariable and Multivariable analysis of overall survival in all treated patients based on
patient characteristics.

| | Univariable | | | Multivariable | | |
|---|---|---|---|---|---|---|
| Characteristics | HR | 95% CI | p-value | HR | 95% CI | p-value |
| Sex | | | 0.092 | | | 0.96 |
| Female | Ref | | | Ref | | |
| Male | 0.59 | 0.31-1.09 | | 0.98 | 0.50-1.97 | |
| Number of prior lines of therapy | 1.19 | 1.01-1.41 | 0.050 | 1.16 | 0.97-1.38 | 0.11 |
| Time dx to infusion (months) | 0.99 | 0.98-1.00 | 0.14 | | | |
| Age at diagnosis | | | 0.004 | | 0.21-0.81 | 0.008 |
| <1 or >10 years | Ref | | | Ref | | |
| 1 to 10 years | 0.39 | 0.20-0.75 | | 0.41 | | |
| Pre-infusion disease burden | Ref | | <0.001 | Ref | | <0.001 |
| No or low disease | 4.76 | 2.10-10.8 | | 4.77 | 2.10-10.9 | |
| High disease | | | | | | |
| Initial cytogenetic risk | | | 0.15 | | | |
| Favorable | Ref | | | | | |
| Intermediate | 3.06 | 0.87-10.8 | | | | |
| Unfavorable | 2.40 | 0.69-8.32 | | | | |
| Prior HCT | | | 0.90 | | | |
| No | Ref | | | | | |
| Yes | 1.09 | 0.26-4.54 | | | | |
| Prior CD19 directed therapy | | | 0.69 | | | |
| No | Ref | | | | | |
| Yes | 1.16 | 0.57-2.38 | | | | |
| Prior relapse | | | 0.065 | | | 0.055 |
| 1 or more relapse | Ref | | | Ref | | |
| Refractory | 0.38 | 0.12-1.24 | | 0.36 | 0.11-1.18 | |
| CNS or EM disease | | | 0.64 | | | |
| No | Ref | | | | | |
| Yes | 0.86 | 0.46-1.62 | | | | |
| Race | | | 0.32 | | | |
| Black/Hispanic | Ref | | | | | |
| Others | 0.68 | 0.16-2.88 | | | | |
| White | 0.62 | 0.32-1.17 | | | | |
| Cumulative Flu AUC (mg * hr/L) | | | 0.22 | | | 0.26 |
| ≥13.8 | Ref | | | Ref | | |
| <13.8 | 1.51 | 0.79-2.87 | | 1.53 | 0.73-3.21 | |

HR, hazard ratio;
CI, confidence interval;
dx, diagnosis;
HCT, hematopoietic cell transplantation;
CNS, central nervous system;
EM, extramedullary disease;
flu, fludarabine;
AUC, area under the curve;
ref, reference

TABLE 6
Univariable analysis of disease characteristics associated
with response and fludarabine exposure.

| | Sub-optimal | | Optimal | | |
|---|---|---|---|---|---|
| | (<13.8 mg*hr/L) | | (≥13.8 mg*hr/L) | | |
| Characteristics | n = 50 | | n = 102 | | p-value |
| Race | | | | | 0.92 |
| Black/Hispanic | 21 | (32%) | 44 | (68%) | |
| Others | 2 | (25%) | 6 | (75%) | |
| White | 26 | (34%) | 50 | (66%) | |
| Unknown | 1 | | 2 | | |
| Pre-infusion disease | | | | | 0.16 |
| burden | | | | | |
| No or low disease | 29 | (39%) | 45 | (61%) | |
| High disease | 20 | (27%) | 54 | (73%) | |
| Unknown | 1 | | 3 | | |
| Prior CD19 directed | | | | | 0.79 |
| therapy | | | | | |
| no | 38 | (32%) | 81 | (68%) | |
| yes | 12 | (36%) | 21 | (64%) | |

Fludarabine Exposure and Toxicity. CRS and severe CRS (sCRS; defined as grade 3 or higher) were observed in 64% (97/152) of patients and in 22% (33/152) of patients, respectively. Neurotoxicity was seen in 24% (36/152) of patients and severe neurotoxicity (defined as grade 3 or higher) in 8% 12/152) of patients. The neurotoxicity grading was heterogeneous with ICANS (n=98), CRES (n=21), and other grading systems (n=22). Optimal fludarabine exposure was not associated with increased CRS or neurotoxicity (limited to ICANS graded patients) (Table 7). Patients who received optimal fludarabine exposure had sCRS of 18% (18/102) compared to 30% (15/50) of patients in the sub-optimal fludarabine exposure group (p=0.10). There was also no difference between optimal and sub-optimal fludarabine exposure and overall ICANS (22% (15/67) vs 29% (9/31), p=0.61) or sICANS (7% (5/67) vs 13% (4/31), p=0.46) respectively (Table 7). Infection post CAR T cell therapy occurred in 38% of patients (57/152) including 35% (36/102) and 42% (21/50) in patients who received optimal versus suboptimal fludarabine exposure respectively (p=0.42). In this cohort, four patients died from infection, all of whom received suboptimal fludarabine. Grade 4 neutropenia was seen in 66% (97/148) of the patients, 67% of patients receiving suboptimal fludarabine, and 65% of patients receiving optimal fludarabine (p=0.84). In addition, there was no correlation between receiving an optimal fludarabine exposure and resolution of neutropenia (p=0.75) or with the time to recovery (in patients who recovered, p=0.61).

TABLE 7
Toxicity rates according to optimal
and sub-optimal fludarabine exposure.

| | Sub-optimal flu | | Optimal flu | | |
|---|---|---|---|---|---|
| Characteristics | (<13.8 mg*hr/L) | | (≥13.8 mg*hr/L) | | p-value |
| CRS grade ≥3 | 15/50 | (30%) | 18/102 | (18%) | 0.09 |
| (sCRS) | | | | | |
| ICANS grade ≥1 | 9/31 | (29%) | 15/67 | (22%) | 0.61 |
| ICANS grade ≥3 | 4/31 | (13%) | 5/67 | (7%) | 0.46 |
| (sICANS) | | | | | |

Flu, fludarabine; CRS, cytokine release syndrome; sCRS, severe CRS.

### Discussion

Lymphodepleting chemotherapy prior to CAR T cell therapy has been shown to be beneficial for efficacy with the current generation of investigational or commercial CAR T cells (Gardner et al., Blood. 06 2017; 129 (25):3322-3331; Curran et al., Blood. Oct. 17, 2019; Turtle et al., J Clin Invest. 06 2016; 126 (6):2123-38; and Hirayama et al., Blood. Apr. 25, 2019; 133 (17):1876-1887). Using a validated population PK model, it was found that fludarabine exposure is a major driver of outcomes in the standard cyclophosphamide and fludarabine LDC prior to tisagenlecleucel for R/R B-ALL in a real-world setting. The presently disclosed subject matter defines an optimal fludarabine exposure for CAR T cell therapy. Optimal fludarabine exposure was found to be ≥13.8 mg*hr/L and was associated with reduced disease relapse and a clinically relevant composite endpoint of relapse or loss of BCA. Fludarabine exposure was noted to impact OS in patients with high pre-infusion tumor burden, a cohort of patients previously reported to have dismal outcomes with CAR T cell therapy (Schultz et al., Blood. 2020; 136 (Supplement 1):14-15). Fludarabine exposure as part of LDC is an easily modifiable factor that could improve the durability and efficacy of CAR T cell therapy.

Higher intensity LDC has been shown to improve lymphodepletion and has been associated with improved response following CD19-specific CAR T cells (Gardner et al., Blood. 06 2017; 129 (25):3322-3331). Other potential mechanisms include increased availability of cytokines and/or improvement of cytokine milieu which has also been shown following more intense more lymphodepletion (Hirayama et al., Blood. Apr. 25, 2019; 133 (17):1876-1887; and Gattinoni et al., J Exp Med. Oct. 3, 2005; 202 (7):907-12). A consideration in personalizing fludarabine dosing is to determine if any increase in toxicity is seen with increasing fludarabine LDC. Previous studies have noted the association of delayed neurotoxicity in adult leukemia patients treated with fludarabine (Warrell et al., J Clin Oncol. January 1986; 4 (1):74-9; Spriggs et al., Cancer Res. November 1986; 46 (11):5953-8; and Chun et al., Cancer Treat Rep. October 1986; 70 (10):1225-8). No increase in toxicity was noted in the current analysis. In this cohort, no apparent increased infection risk was noted in patients who received increased fludarabine, e.g., ≥13.8 mg*hr/L, exposure.

The use of a fludarabine population PK model is clinically feasible, and unique data on the association of fludarabine exposure and outcomes following CAR T cell therapy were generated. Patients also received cyclophosphamide as part of their LDC, however cyclophosphamide pharmacokinetics are not included in this analysis as the number of active metabolites of cyclophosphamide make estimation of exposure not feasible. This analysis can be applied in alternative LDC agents that have more predictable PK thereby creating a more favorable environment for CAR T cell expansion, persistent, and durability.

In summary, increased, e.g., ≥13.8 mg*hr/L, fludarabine exposure prior to CD19-specific CAR T cell therapy (tisagenlecleucel) in pediatric and young adult patients with R/R B-ALL was associated with lower relapse.

Although the presently disclosed subject matter and certain of its advantages have been described in detail, it should be understood that various changes, substitutions and alterations can be made herein without departing from the spirit and scope of the disclosure. Moreover, the scope of the present application is not intended to be limited to the particular embodiments of the process, machine, manufacture, and composition of matter, and methods described in the specification. As one of ordinary skill in the art will readily appreciate from the disclosure of the presently disclosed subject matter, processes, machines, manufacture, compositions of matter, or methods, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, or methods.

Various patents, patent applications, publications, product descriptions, protocols, and sequence accession numbers are cited throughout this application, the disclosure of which are incorporated herein by reference in their entireties for all purposes.

What is claimed is:

1. A method of treating a subject, increasing durability of a therapy, improving efficacy of a therapy, lowering the risk of relapse, and/or lengthening survival comprising administering to the subject cells comprising an antigen recognizing receptor, wherein, prior to the administration, the subject has been preconditioned with a personalized amount of a chemotherapeutic agent, wherein the personalized amount provides an area under the curve (AUC) of at least about 10 mg·hr/L of the chemotherapeutic agent.

2. The method of claim 1, wherein the personalized amount of the chemotherapeutic agent provides an AUC of at least about 11 mg·hr/L, at least about 12 mg·hr/L, at least about 13 mg·hr/L, at least about 14 mg·hr/L, or at least about 15 mg·hr/L of the chemotherapeutic agent.

3. The method of claim 1, wherein the personalized amount of the chemotherapeutic agent provides an AUC of at least about 13 mg·hr/L of the chemotherapeutic agent or an AUC of about 14 mg·hr/L of the chemotherapeutic agent.

4. The method of claim 1, wherein the personalized amount is determined by body weight and renal function of the subject.

5. The method of claim 4, wherein the renal function is determined by a glomerular filtration rate (GFR) of the subject.

6. The method of claim 5, wherein the GFR is determined by creatinine clearance of the subject.

7. The method of claim 1, wherein the chemotherapeutic agent is an antimetabolite, an adenosine deaminase inhibitor, or a purine analog.

8. The method of claim 7, wherein
a) the antimetabolite is selected from the group consisting of folic acid antagonists, pyrimidine analogs, purine analogs, adenosine deaminase inhibitors, lympho- or myeloid-depleting antibodies, alkylators, topoisomerase II inhibitors, derivatives thereof, and combinations thereof;
b) the adenosine deaminase inhibitor is fludarabine or a derivative thereof; and
c) the purine analog is clofarabine or a derivative thereof.

9. The method of claim 1, wherein the subject suffers a tumor or a cancer.

10. The method of claim 9, wherein the tumor is blood cancer, B-cell malignancy, leukemia, or lymphoma.

11. The method of claim 9, wherein the tumor is selected from the group consisting of B cell leukemia, B cell lymphoma, acute lymphoblastic leukemia (ALL), chronic lymphocytic leukemia (CLL), non-Hodgkin's lymphoma, Burkitt lymphoma, acute myeloid leukemia (AML) and Mixed-phenotype acute leukemia (MPAL).

12. The method of claim 11, wherein the tumor is B cell acute-lymphoblastic leukemia (B-ALL).

13. The method of claim 1, wherein the subject has a high disease burden at the time of or immediately prior to the preconditioning.

14. The method of claim 13, wherein the subject who has high disease burden has more than about 5% lymphoblasts in bone marrow, detectable peripheral blood lymphoblasts in the subject, a CNS3 status, and/or non-CNS extramedullary (EM) site of disease

15. The method of claim 14, wherein the CNS3 status is determined by detecting at least about 5 white blood cells/ml cerebrospinal fluid and at least one lymphoblast in the cerebrospinal fluid.

16. The method of claim 1, wherein the antigen-recognizing receptor is a T cell receptor (TCR) or a chimeric antigen receptor (CAR).

17. The method of claim 1, wherein the antigen-recognizing receptor is a CAR comprising an extracellular antigen-recognition domain that binds to a tumor antigen and an intracellular signaling domain.

18. The method of claim 17, wherein
a) the tumor antigen is CD19;
b) the intracellular signaling domain comprises a CD3 polypeptide; and/or
c) the intracellular signaling domain comprises a CD3 polypeptide and at least one co-stimulatory signaling region.

19. The method of claim 18, wherein the costimulatory signaling region comprises an intracellular domain of 4-1BB or an intracellular domain of CD28.

20. The method of claim 1, wherein
a) the cell is an immunoresponsive cell;
b) the cell is a cell of the lymphoid lineage or a cell of the myeloid lineage;
c) the cell is selected from the group consisting of T cells, Natural Killer (NK) cells, stem cells from which lymphoid cells may be differentiated;
d) the cell is selected from the group consisting of T cells, Natural Killer (NK) cells, stem cells from which lymphoid cells may be differentiated;
e) the cell is a T cell;
f) the cell is a T cell selected from the group consisting of a cytotoxic T lymphocyte (CTL), a γδ T cell, a tumor-reactive lymphocyte, a tumor-infiltrating lymphocyte (TIL), a regulatory T cell, and a Natural Killer T (NKT) cell.
